(19) United States
(12) Patent Application Publication
(10) Pub. No.: US 2014/0256760 A1
(43) Pub. Date: Sep. 11, 2014

(54) REDUCTION OR PREVENTION OF ALCOHOL REACTION WITH DIETARY SUPPLEMENTS

(71) Applicant: NOGLO LLC, SAN FRANCISCO, CA (US)

(72) Inventors: Robert James TOLLETH, Millbrae, CA (US); Spencer Adam GORDON, Ramona, CA (US)

(73) Assignee: NOGLO LLC, San Francisco, CA (US)

(21) Appl. No.: 14/150,705

(22) Filed: Jan. 8, 2014

Related U.S. Application Data
(63) Continuation of application No. PCT/US2013/067345, filed on Oct. 29, 2013.
Application No. PCT/US2013/067345 is a continuation-in-part of application No. 13/938,079, filed on Jul. 9, 2013.
Application No. 13/938,079 is a continuation-in-part of application No. PCT/US2013/049783, filed on Jul. 9, 2013.
(60) Provisional application No. 61/780,612, filed on Mar. 13, 2013.
(60) Provisional application No. 61/780,612, filed on Mar. 13, 2013.
(60) Provisional application No. 61/669,404, filed on Jul. 9, 2012.
(60) Provisional application No. 61/780,612, filed on Mar. 13, 2013.
(60) Provisional application No. 61/669,404, filed on Jul. 9, 2012.
(60) Provisional application No. 61/780,612, filed on Mar. 13, 2013.

(51) Int. Cl. A61K 31/455; A61K 31/198; A61K 45/06; A61K 31/375; A61K 31/385; A61K 31/51; A61K 31/197; A61K 31/07

(52) U.S. Cl. CPC A61K 31/455; A61K 31/197; A61K 31/198; A61K 31/07; A61K 31/375; A61K 31/385; A61K 31/51; A61K 45/06
USPC 514/276; 514/355

(57) ABSTRACT

The present invention is directed to compositions for preventing or reducing the flush and other unpleasant symptoms that sometimes accompany the consumption of alcoholic beverages, and methods for use of said compositions. The compositions comprise an agent, or combinations of various agents, such as an agent capable of increasing intracellular or intramitochondrial concentration of NAD in the subject; an agent capable of increasing glutathione concentration in the subject; an anti-oxidant; alpha-lipoic acid or a precursor to alpha-lipoic acid; and/or an agent capable of increasing the level of Coenzyme A in the subject; where the agent or agents are present or administered in amounts effective to prevent or reduce the alcohol reaction in a subject who has consumed one or more alcoholic beverages.

### CROSS-REFERENCE TO RELATED APPLICATIONS

This application claims priority benefit of U.S. Provisional Patent Application No. 61/780,612, filed Mar. 13, 2013, and is a continuation of and claims priority benefit of International Patent Application No. PCT/US2013/067345 filed Oct. 29, 2013, which claims priority benefit of U.S. Provisional Patent Application No. 61/780,612, filed Mar. 13, 2013. This application is a continuation-in-part of and claims priority benefit of pending U.S. patent application Ser. No. 13/938,079 filed Jul. 9, 2013, which in turn claims priority benefit of U.S. Provisional Patent Application No. 61/669,404 filed Jul. 9, 2012 (expired) and priority benefit of U.S. Provisional Patent Application No. 61/780,612, filed Mar. 13, 2013. This application is a continuation-in-part of and claims priority benefit of pending International Patent Application No. PCT/US2013/049783 filed Jul. 9, 2013, which in turn claims priority benefit of U.S. Provisional Patent Application No. 61/669,404 filed Jul. 9, 2012 (expired) and priority benefit of U.S. Provisional Patent Application No. 61/780,612, filed Mar. 13, 2013. The entire contents of each of those applications are hereby incorporated by reference herein.

### TECHNICAL FIELD OF THE INVENTION

The present invention relates to compositions and methods for reducing or preventing the flush and other unpleasant symptoms that sometimes accompany the consumption of alcoholic beverages.

### BACKGROUND OF THE INVENTION

The alcohol reaction, also known as the alcohol flush reaction, is an unpleasant condition which occurs in some individuals upon consumption of alcohol, even in relatively small amounts. The reaction is characterized by flushing of the facial skin, and sometimes other areas of the skin. The alcohol reaction may also include dizziness, nausea, stupor, headache, light-headedness, increased heart rate, and other symptoms.

The alcohol reaction occurs in individuals with lower rates of metabolism of acetaldehyde. Acetaldehyde is metabolized by acetaldehyde dehydrogenase-2 (ALDH2; also known as mitochondrial aldehyde dehydrogenase; human precursor Swiss-Prot PO5091), a liver enzyme involved in the metabolism of ethanol, which oxidizes acetaldehyde to acetic acid. Approximately 1 billion people, including up to about 50% of people of Chinese, Japanese and Korean ethnicity, have a point mutation resulting in a less active version of ALDH2 (ALDH2*2, where a glutamic acid residue is mutated to lysine, i.e., ALDH2 E487K; the residue numbering is based on the mature protein; see, e.g., Steinmetz et al., Structure 5: 701-11, 1997, which is hereby incorporated by reference in its entirety), which functions at about 8% of the wild-type form. The enzyme is normally a homotetramer. However, if an individual carries one mutated allele, then he or she will present the mutant phenotype due to the formation of heterotetramers. The mutation in ALDH2 is at an amino acid residue located at the binding site for nicotinamide adenine dinucleotide (NAD), an enzyme cofactor required for the conversion of acetaldehyde to acetic acid; in the process, NAD is converted to NADH. The lowered activity of this enzyme results in an accumulation of acetaldehyde in the blood when consuming alcohol. Certain individuals, especially those of East Asian descent, may also have a highly active variant of alcohol dehydrogenase (ADH1B; also known as alcohol dehydrogenase 1B; human precursor Swiss-Prot P00325) caused by a R47H mutation (again, this residue numbering is based on the mature protein), leading to more rapid accumulation of acetaldehyde (Peng et al., BMC Evolutionary Biology 2010, 10:15). Individuals with both the “fast” variant of alcohol dehydrogenase (which produces acetaldehyde from alcohol) and the “slow” variant of acetaldehyde dehydrogenase (which eliminates acetaldehyde) can be strongly affected by the alcohol reaction.

Acetaldehyde is 30 times as toxic as ethanol and is a known carcinogen. In addition to causing inflammation and flushing of the skin, acetaldehyde can cause organ damage. There is evidence demonstrating that individuals with the variant ALDH2 enzyme are at a significantly increased risk of gastric and esophageal cancers, liver cirrhosis and failure, and even Alzheimer's disease if they consume alcohol with any regularity. This is shown to be due to the accumulation of acetaldehyde. Individuals that demonstrate the flush response to alcoholic beverages and drink moderately are at a much higher risk of esophageal cancer than even heavy drinkers who do not experience the flush reaction. In addition, drinkers that flush may also experience dizziness and nausea after just one drink.

The health risks associated with drinking and flushing are not well understood by the general public. The majority of research on the ALDH2 mutation and alcohol related diseases has been done in Japan, and the results of this research are not as widely disseminated in other countries, including the United States, which in 2009 had an estimated 4 million residents of Chinese descent, 1.6 million residents of Korean descent, 1.3 million residents of Japanese descent, and several million additional residents from other East Asian countries. While awareness of the health risks associated with the alcohol reaction is increasing in Japan and China, people continue to drink regularly despite these known long term risks.

Thus, there is a need for compositions and methods for prevention or reduction of the alcohol reaction, both for the short-term comfort of the alcohol consumer, and for the potential to reduce the long-term health risks associated with consumption of alcoholic beverages by persons demonstrating the alcohol reaction.

### SUMMARY OF THE INVENTION

In some embodiments, the invention provides compositions and methods for prevention or reduction of the alcohol reaction in a subject.

In some embodiments, the invention embraces a composition for use by a subject to prevent or reduce the alcohol reaction and/or to reduce acetaldehyde in tissues and body fluids (e.g., liver, central nervous system, blood, saliva, etc.). The composition comprises an agent capable of increasing intracellular or intramitochondrial concentration of NAD in the subject, and an agent capable of increasing glutathione concentration in the subject, where the agents are present in the composition in an amount sufficient to prevent or reduce the alcohol reaction in the subject. In additional embodiments, the composition can further comprise one or more of the following:

an agent capable of increasing the level of Coenzyme A in the subject;

an agent which is an anti-oxidant;

alpha-lipoic acid or a precursor to alpha-lipoic acid;

an agent capable of increasing the level of Coenzyme A in the subject and an anti-oxidant;

an agent capable of increasing the level of Coenzyme A in the subject and alpha-lipoic acid or a precursor to alpha-lipoic acid;

an anti-oxidant and alpha-lipoic acid or a precursor to alpha-lipoic acid; or

an agent capable of increasing the level of Coenzyme A in the subject, an anti-oxidant, and alpha-lipoic acid or a precursor to alpha-lipoic acid. This list is not meant to be limiting.

In some embodiments, the invention embraces a composition for use by a subject to prevent or reduce the alcohol reaction and/or to reduce acetaldehyde in tissues and body fluids, where the composition comprises an agent capable of increasing intracellular or intramitochondrial concentration of NAD in the subject. In some embodiments, the invention embraces a composition for use by a subject to prevent or reduce the alcohol reaction, where the composition comprises an agent capable of increasing glutathione concentration in the subject. In some embodiments, the invention embraces a composition for use by a subject to prevent or reduce the alcohol reaction, where the composition comprises an agent capable of increasing intracellular Coenzyme A concentration in the subject. In these embodiments, the agent is present in the composition in an amount sufficient to prevent or reduce the alcohol reaction in the subject. The agent can be administered to the subject in a daily dosage for at least one week prior to consumption of alcohol. The daily dosage can be administered in a single dosage per day, or in multiple divided dosages per day.

In some embodiments, the invention embraces a composition for use by a subject to prevent or reduce the alcohol reaction and/or to reduce acetaldehyde in tissues and body fluids. The composition can be described as an anti-alcohol reaction supplement. The supplement or composition comprises an agent capable of increasing intracellular or intramitochondrial concentration of NAD in the subject; an agent capable of increasing glutathione concentration in the subject; an agent which is an anti-oxidant; an agent which is alpha-lipoic acid or a precursor to alpha-lipoic acid; and an agent capable of increasing the level of Coenzyme A in the subject The agents are present in the supplement or composition in an amount or amounts effective to prevent or reduce the alcohol reaction in the subject. In other embodiments, the agents are present in the supplement or composition in an amount or amounts effective to prevent the alcohol reaction in the subject. In other embodiments, the agents are present in the supplement or composition in an amount or amounts effective to reduce the alcohol reaction in the subject.

The agent capable of increasing intracellular or intramitochondrial concentration of NAD in the subject can be nicotinamide, niacin, nicotinamide riboside, or oxaloacetate. If nicotinamide is used, the amount of nicotinamide in the supplement or composition can range from about 500 mg to about 1,500 mg, or from about 750 mg to about 1,250 mg. In another embodiment, the amount of nicotinamide in the supplement or composition is about 1000 mg. In another embodiment, the amount of nicotinamide in the supplement or composition is about 800 mg. If niacin is used, the amount of niacin in the supplement or composition can range from about 500 mg to about 1,500 mg, or from about 750 mg to about 1,250 mg. In another embodiment, the amount of niacin in the supplement or composition is about 1000 mg. In another embodiment, the amount of niacin in the supplement or composition is about 800 mg. If nicotinamide riboside is used, the amount of nicotinamide riboside in the supplement or composition can range from about 1,000 mg to about 3,200 mg, or from about 1,500 mg to about 2,600 mg. In another embodiment, the amount of nicotinamide riboside in the supplement or composition is about 2,100 mg. In another embodiment, the amount of nicotinamide riboside in the supplement or composition is about 1,700 mg.

The agent capable of increasing glutathione concentration in the subject can be N-acetyl cysteine, L-cysteine Vitamin C, alpha lipoic acid, glutathione, glycine, L-glutamic acid, adenosylmethionine, or conjugated linoleic acid. If N-acetyl cysteine is used, the amount in the supplement or composition can range from about 200 mg to about 600 mg, or from about 300 mg to about 500 mg. In another embodiment, the amount of N-acetyl cysteine in the supplement or composition is about 400 mg. In another embodiment, the amount of N-acetyl cysteine in the supplement or composition is about 300 mg. If L-cysteine is used, the amount in the supplement or composition can range from about 200 mg to about 600 mg, or from about 300 mg to about 500 mg. In another embodiment, the amount of L-cysteine in the supplement or composition is about 400 mg. In another embodiment, the amount of L-cysteine in the supplement or composition is about 300 mg.

The agent used as the anti-oxidant can be Vitamin C, Vitamin E, or alpha-lipoic acid. If Vitamin C is used, the amount in the supplement or composition can range from about 100 mg to about 500 mg, or from about 200 mg to about 400 mg. In another embodiment, the amount of Vitamin C in the supplement or composition is about 300 mg. In another embodiment, the amount of Vitamin C in the supplement or composition is about 150 mg.

The amount of alpha lipoic acid in the supplement or composition, when a separate anti-oxidant is used in addition to alpha lipoic acid, can range from about 50 mg to about 250 mg, or from about 100 mg to about 200 mg. In one embodiment, the amount of alpha-lipoic acid in the supplement or composition is about 150 mg. In one embodiment, the amount of alpha-lipoic acid in the supplement or composition is about 100 mg. If alpha lipoic acid is also used as the anti-oxidant, the amount of alpha lipoic acid in the supplement or composition can range from about 100 mg to about 500 mg, from about 200 mg to about 400 mg, or in another embodiment is about 300 mg, or in another embodiment is about 200 mg.

The agent capable of increasing the level of Coenzyme A in the subject can be pantothenic acid, pantothenate, panthenol, pantethine, pantetheine, 4′-phosphopantothenate, 4′-phosphopantetheine, N-acetyl-cysteine, or L-cysteine. If pantothenic acid is used, the amount of pantothenic acid in the supplement or composition can range from about 50 mg to about 250 mg, or from about 100 mg to about 200 mg. In another embodiment, the amount of pantothenic acid in the supplement or composition is about 150 mg. In another embodiment, the amount of pantothenic acid in the supplement or composition is about 100 mg. If pantothenate is used, the amount of pantothenate in the supplement or composition can range from about 50 mg to about 250 mg, or from about 100 mg to about 200 mg. In another embodiment, the amount of pantothenate in the supplement or composition is about 150 mg. In another embodiment, the amount of pantothenate in the supplement or composition is about 100 mg. If panthenol is used, the amount of panthenol in the supplement or composition can range from about 50 mg to about 250 mg, or from about 100 mg to about 200 mg. In another embodiment, the amount of panthenol in the supplement or composition is about 150 mg. In another embodiment, the amount of panthenol in the supplement or composition is about 100 mg.

In certain embodiments, the present invention provides compositions which comprise (i) between 300 mg and 1.5 g nicotinamide, (ii) between 10 mg and 500 mg pantothenic acid or a salt thereof, and (iii) between 100 mg and 600 mg N-acetylcysteine. In additional embodiments, the present invention provides compositions which comprise (i) between 500 mg and 900 mg nicotinamide, (ii) between 25 mg and 75 mg pantothenic acid or a salt thereof, and (iii) between 200 mg and 400 mg N-acetylcysteine. In preferred embodiments, the compositions comprise about 700 mg nicotinamide, about 50 mg pantothenic acid, and about 300 mg N-acetylcysteine; or about 700 mg nicotinamide, about 25 mg pantothenic acid, and about 300 mg N-acetylcysteine; or about 700 mg nicotinamide, about 100 mg pantothenic acid, and about 300 mg N-acetylcysteine; wherein the term “about” refers to +/−20% of each quantity. By way of example, the present invention can comprise a daily dosage of 700 mg nicotinamide, 50 mg pantothenic acid, and 300 mg N-acetylcysteine, or a daily dosage of 700 mg nicotinamide, 25 mg pantothenic acid, and 300 mg N-acetylcysteine, or a daily dosage of 700 mg nicotinamide, 100 mg pantothenic acid, and 300 mg N-acetylcysteine. These compositions can further comprise one or more of vitamin A, vitamin C, thiamine, and α-lipoic acid, in addition to the nicotinamide, pantothenic acid (or salt thereof), and N-acetylcysteine. This list of further components is not meant to be limiting. In some embodiments, the compositions further comprise, in addition to the nicotinamide, pantothenic acid (or salt thereof), and N-acetylcysteine, one or more of, and preferably each of, 1250 IU to 37500 IU vitamin A, 75 mg to 250 mg vitamin C, 2.5 to 100 mg thiamine, and 50 to 125 mg α-lipoic acid. In further embodiments, the compositions further comprise, in addition to the nicotinamide, pantothenic acid (or salt thereof), and N-acetylcysteine, one or more of, and preferably each of, 1250 IU to 2500 IU vitamin A, 75 mg to 150 mg vitamin C, 2.5 to 10 mg thiamine, and 62 to 125 mg α-lipoic acid. In further embodiments, the compositions comprise (i) between 300 mg and 1.5 g nicotinamide, (ii) between 10 mg and 500 mg pantothenic acid or a salt thereof, (iii) between 100 mg and 600 mg N-acetylcysteine, (iv) 1250 IU to 37500 IU vitamin A, (v) 75 mg to 250 mg vitamin C, (vi) 2.5 to 100 mg thiamine, and (vii) 50 to 125 mg α-lipoic acid. In further embodiments, the compositions comprise (i) between 500 mg and 900 mg nicotinamide, (ii) between 25 mg and 75 mg pantothenic acid or a salt thereof, (iii) between 200 mg and 400 mg N-acetylcysteine, (iv) 1250 IU to 37500 IU vitamin A, (v) 75 mg to 250 mg vitamin C, (vi) 2.5 to 100 mg thiamine, and (vii) 50 to 125 mg α-lipoic acid. In preferred embodiments, the compositions comprise about 700 mg nicotinamide, about 50 mg pantothenic acid, about 300 mg N-acetylcysteine, about 2500 IU vitamin A, about 150 mg vitamin C, about 50 mg thiamine, and about 80 mg α-lipoic acid wherein the term “about” refers to +/−20% of each quantity. By way of example, the compositions can comprise 700 mg nicotinamide, 50 mg pantothenic acid, 300 mg N-acetylcysteine, 2500 IU vitamin A, 150 mg vitamin C, 50 mg thiamine, and 80 mg α-lipoic acid.

As noted herein, such daily dosages can be taken as a single administration, or in 2, 3, 4, 5 or more individual administrations. The skilled artisan will understand that the components of the composition may be provided as various salt or prodrug forms. By way of example only, pantothenic acid may be provided in the form of calcium pantothenate.

Preferred are compositions which comprise one or more pharmaceutical excipients acceptable for oral or enteral administration. The compositions may be provided as a tablet or capsule; as a beverage; as a food; etc.

The present invention can comprise establishment of a daily dose over a period of time, e.g. 3 days, 5 days, 10 days, 14 days, 21 days, 28 days, 30 days, or longer, and administering a supplemental dosage between 10 minutes and 1 hour prior consumption of ethanol by the human adult.

As described herein, the levels of the compositions of the present invention can reduce average peak blood levels of acetaldehyde in a population of human adults having an E487K mutation in mitochondrial aldehyde dehydrogenase and/or a R47H mutation in alcohol dehydrogenase 1B following ingestion of 0.5 g ethanol/kg body wt by at least 5%, more preferably 10%, still more preferably 20%, yet more preferably 40%, and most preferably 50% or more relative to average peak blood levels in a corresponding untreated population. In further embodiments, the levels of the compositions of the present invention can reduce average peak blood levels of acetaldehyde in a human adult having an E487K mutation in mitochondrial aldehyde dehydrogenase and/or a R47H mutation in alcohol dehydrogenase 1B following ingestion of 0.5 g ethanol/kg body wt by at least 5%, more preferably 10%, still more preferably 20%, yet more preferably 40%, and most preferably 50% or more relative to average peak blood levels in the same individual absent treatment.

In another embodiment, the invention embraces a method of preventing or reducing at least one symptom of the alcohol reaction in a subject, by administering the supplement or composition as described in any of the foregoing embodiments. The dosage of the supplement or composition can be administered to the subject daily, either in a single dose per day, or in multiple divided doses per day. The composition is administered prior to the consumption of alcohol, and can be administered daily for a period of time such as at least seven days prior to consumption of alcohol by the subject, at least 14 days prior to consumption of alcohol by the subject, or at least 30 days prior to consumption of alcohol by the subject.

In another embodiment, the invention embraces a method of preventing or reducing at least one symptom of the alcohol reaction in a subject, by administering to the subject at least one agent selected from the group consisting of an agent capable of increasing intracellular or intramitochondrial concentration of NAD in the subject, an agent capable of increasing glutathione concentration in the subject, and an agent capable of increasing cellular Coenzyme A concentrations in the subject, where the at least one agent is administered in an amount sufficient to prevent or reduce the alcohol reaction in the subject. The dosage of the agent or agents can be administered to the subject daily, either in a single dose per day, or in multiple divided doses per day. The composition is administered prior to the consumption of alcohol, and can be administered daily for a period of time such as at least seven days prior to consumption of alcohol by the subject, at least 14 days prior to consumption of alcohol by the subject, or at least 30 days prior to consumption of alcohol by the subject.

The symptom or symptoms to be prevented or reduced are selected from facial flushing, hangover, headache, stupor, dizziness, nausea, accumulation of acetaldehyde in the blood (i.e., increased levels of blood acetaldehyde), or increased heart rate. In some embodiments, the symptom to be reduced is facial flushing. In another embodiment, the symptom to be reduced is hangover. In another embodiment, the symptom to be reduced is headache. In another embodiment, the symptom to be reduced is stupor. In another embodiment, the symptom to be reduced is dizziness. In another embodiment, the symptom to be reduced is nausea. In another embodiment, the symptom to be reduced is accumulation of acetaldehyde in the blood or increased levels of blood acetaldehyde. In another embodiment, the symptom to be reduced is increased heart rate. In another embodiment, the symptoms to be reduced are any combination of any or all of the foregoing symptoms. In some embodiments, the symptom to be prevented is facial flushing. In another embodiment, the symptom to be prevented is hangover. In another embodiment, the symptom to be prevented is headache. In another embodiment, the symptom to be prevented is stupor. In another embodiment, the symptom to be prevented is dizziness. In another embodiment, the symptom to be prevented is nausea. In another embodiment, the symptom to be prevented is accumulation of acetaldehyde in the blood. In another embodiment, the symptom to be prevented is increased heart rate. In another embodiment, the symptoms to be prevented are any combination of any or all of the foregoing symptoms.

In additional embodiments, the invention includes a method of reducing the accumulation of acetaldehyde in the blood after consumption of alcohol, by administering the supplement or composition of the invention. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 5%, up to about 5%, or by at least about 5% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 10%, up to about 10%, or by at least about 10% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 15%, up to about 15%, or by at least about 15% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 20%, up to about 20%, or by at least about 20% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 25%, up to about 25%, or by at least about 25% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 50%, up to about 50%, or by at least about 50% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 75%, up to about 75%, or by at least about 75% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 90%, up to about 90%, or by at least about 90% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol. In some embodiments, the invention includes a method of reducing the accumulation of acetaldehyde in the blood after consumption of alcohol, by administering the supplement or composition of the invention in an amount and duration sufficient to reduce the accumulation of acetaldehyde by about 95%, up to about 95%, or by at least about 95% from the blood concentration that would otherwise be reached without administration of the supplement or composition. The level of acetaldehyde can be measured 10 minutes after the consumption of alcohol, 20 minutes after the consumption of alcohol, or 40 minutes after the consumption of alcohol, preferably 20 minutes after the consumption of alcohol.

In additional embodiments, the invention includes the use of any of the compositions described herein for prevention or reduction of at least one symptom of the alcohol reaction in a subject. In additional embodiments, the invention includes the use of any of the compositions described herein for the manufacture of a medicament for use in preventing or reducing at least one symptom of the alcohol reaction in a subject. In additional embodiments, the invention includes the use of any of the compositions described herein for the manufacture of a dietary supplement for use in preventing or reducing at least one symptom of the alcohol reaction in a subject.

In any of the embodiments of the compositions, supplements, methods, or uses described herein, the composition or supplement can further comprise an agent capable of raising the blood level of all-trans retinoic acid.

The present invention comprises multiple aspects, features and embodiments, where such multiple aspects, features and embodiments can be combined and permuted in any desired manner. These and other aspects, features and embodiments of the present invention will become evident upon reference to the remainder of this application, including the following detailed description.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition is described as “consisting essentially of” the listed components, the composition contains the components expressly listed, and may contain other components which do not substantially affect condition being treated. However, the composition either does not contain any other components which do substantially affect the condition being treated other than those components expressly listed; or, if the composition does contain extra components other than those listed which substantially affect the condition being treated, the composition does not contain a sufficient concentration or amount of those extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed.

The compositions and methods described herein, including any embodiment of the invention as described herein, may be used alone or may be used in combination with other compositions and methods.

Any of the compositions and methods described herein, including any embodiment of the invention as described herein, can be used for reduction of symptoms of the alcohol reaction (that is, reduction, but not complete prevention).

### DETAILED DESCRIPTION OF THE INVENTION

The present invention relates to compositions which are combinations of dietary supplements or compositions useful for prevention or reduction of the alcohol reaction, and methods for use, including dosage regimens, of the compositions to prevent or reduce the alcohol reaction. In some embodiments, the supplement or composition reduces the accumulation of acetaldehyde in the blood after consumption of alcoholic beverages.

A supplement or composition of the invention comprising the combined ingredients can be taken once a day, or in multiple divided dosages over a day, or can be taken as individual components over the course of a day. The supplements or compositions comprise vitamins, amino acids, anti-oxidants, and other dietary supplements. While not wishing to be limited by theory, it is hypothesized that use of the supplements or compositions of the invention results in a protective buildup of compounds which reduces the reactivity of acetaldehyde and which enhances enzyme kinetics, causing an increased rate of breakdown of acetaldehyde. The ALDH2 E487K enzyme variant binds NAD less strongly than the variant with glutamic acid at position 487. Thus, it is hypothesized that increasing NAD concentration will enhance the binding of NAD to aldehyde dehydrogenase, increasing the rate of acetaldehyde dehydrogenation.

Acetaldehyde is oxidized to acetic acid by ALDH2. Acetic acid then binds to Coenzyme A to form Acetyl-CoA and enters the Citric Acid Cycle. While not wishing to be limited by theory, it is hypothesized that during alcohol metabolism in subjects with the ALDH2 mutation, there is a build-up of acetic acid, and Coenzyme A is greatly reduced, as it is not regenerated quickly enough to keep up with the rate of production of acetic acid. With an accumulation of acetic acid, ALDH2 is inhibited based on known enzyme kinetics, as an increase in product concentration will decrease the favorability of the forward reaction. In some embodiments, the invention includes daily supplementation of any compound(s) to increase cellular Coenzyme A concentrations to increase the rate of formation of Acetyl-CoA, therefor clearing acetic acid more quickly and pushing the ALDH2 reaction towards the product. This reduces the accumulation and exposure to acetaldehyde, and as a result, reduces or prevents the alcohol reaction.

### DEFINITIONS

A subject, individual, or patient is a mammal, preferably a human.

Alcohol as used herein refers to ethanol (ethyl alcohol), unless another alcohol is explicitly indicated.

Preventing the alcohol reaction is defined as preventing at least one symptom of the alcohol reaction, such as facial flushing, dizziness, nausea, increased heart rate, or other symptoms in a subject who would ordinarily experience such a reaction absent the use of the compositions and methods of the invention, or of preventing the buildup of acetaldehyde in the blood subsequent to alcohol consumption, that would occur absent the use of the compositions and methods of the invention. Reducing the alcohol reaction is defined as reducing the intensity of at least one symptom of the alcohol reaction, such as facial flushing, dizziness, nausea, increased heart rate, or other symptoms in a subject, or of reducing the buildup of acetaldehyde in the blood subsequent to alcohol consumption, as compared to the intensity of said symptoms in the subject absent the use of the compositions and methods of the invention. Prevention or reduction of the symptoms of the alcohol reaction can be self-reported by the subject (that is, subjective), or reported or measured in the subject for symptoms amenable to observation by others (that is, objective).

The compounds described herein can occur and can be used as the neutral (non-salt) compounds; however, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared. When salts of compounds are used, the weight of the compound(s) used in the compositions and supplements of the invention can be adjusted to take into account the additional weight contributed due to the salt form.

By “supplement” is meant a composition that is taken in addition to the ordinary diet of an individual, subject, or patient. The supplement can be taken with or without food, at any time of day (including divided dosages over the course of a day).

### Measurement and Quantitation of Symptoms

The various symptoms of the alcohol reaction can be self-reported by the subject either qualitatively (for example, flushing or not flushing) or quantitatively (for example, on a scale of one to ten for any of the symptoms; hangovers can be measured using standard scales used to describe pain in clinical settings, such as the Wong-Baker FACES Pain Rating Scale or the 0-10 Numeric Pain Rating Scale described by McCaffery M. and Casero, C. in Pain: Clinical Manual, St. Louis: Mosby, 1999 at p. 16). The symptoms can also be reported or measured by others, again either qualitatively or quantitatively.

Measurements of skin and facial flushing can be quantitated using a chromameter (see, for example, the studies done in Muizzuddin et al., J. Soc. Cosmet. Chem. 41:369-378 (1990) and Alaluf et al., J. Nutr. 132:399-403 (2002)). Chromameters are commercially available from vendors such as Konica Minolta (CR-400 or CR-410 Chroma Meter) or Gigahertz-Optik (HCT-99D color meter). Typically, baseline readings at one or more wavelengths are taken, and the baseline values are subtracted from subsequent readings. Flushing of the skin, especially facial flushing, is a very common symptom of the alcohol reaction, and can be measured non-invasively and in a time-dependent manner.

Blood (plasma or serum) concentrations of acetaldehyde can be readily measured using techniques known in the art (see, for example, McCarver-May, D. et al., Journal of Analytical Toxicology, 21:134 (1997) and Badawy et al., Alcohol and Alcoholism 46:651-660 (2011), which discusses issues in measurement of total whole blood acetaldehyde). This provides an objective measurement of acetaldehyde blood levels and can also be determined in a time-dependent manner. The concentration of acetaldehyde can be measured 10 minutes after the consumption of alcohol, 20 minutes after the consumption of alcohol, or 40 minutes after the consumption of alcohol, preferably 20 minutes after the consumption of alcohol. The concentration of acetaldehyde can also be measured 1 hour after the consumption of alcohol, 2 hours after the consumption of alcohol, or 4 hours after the consumption of alcohol.

### Increase of Biomarkers and Measurement and Quantitation of Biomarkers

The effects of various agents on the levels of various biomarkers, such as Coenzyme A, glutathione, and NAD, can be measured by a variety of techniques. Measurement of biomarker levels can provide an indirect measurement of the degree of protection provided by a particular dosage regimen, and/or can guide a subject in choice of, or modification of, a dosage regimen. Blood levels of biomarkers can be measured in whole blood, in plasma, in serum, or in extracts thereof.

NAD concentration can be measured by extraction from blood samples, and quantitated by enzymatic spectrophotometric methods, such as the procedure described in Majamaa et al., “Increase of Blood NAD+ and Attenuation of Lacticacidemia During Nicotinamide Treatment of a Patient with the MELAS Syndrome,” Life Sciences, volume 58(8): (1996).

Coenzyme A concentrations can be measured by Cayman Coenzyme A Assay Kit f (Cayman Chemical Company, Ann Arbor, Mich., USA; Item Number 700440). Coenzyme A concentrations can be measured in plasma, serum, urine, cell lysates, or tissue homogenates. In one embodiment the invention raises measurable blood Coenzyme A concentrations which are reflective of intracellular concentrations in erythrocytes, or red blood cells (RBCs).

Glutathione concentrations can measured by High-Performance Liquid Chromatography (HPLC) after blood collection, or by colorimetric methods, such as the 5,5′-dithiobis-2-nitrobenzoic acid assay described in Johnston et al., “Vitamin C Elevates Red Blood Cell Glutathione in Healthy Adults,” American Journal of Clinical Nutrition, volume 58(1):103 (1993) and in Beutler et al., “Improved method for the determination of blood glutathione,” J. Lab. Clin. Med. 61:882-888 (1963).

All-trans-retinoic acid concentration can be measured by High Performance Liquid Chromatography (HPLC) after blood collection by various methods, such as those described in De Leenheer et al., “All-trans-retinoic acid: measurement of reference values in human serum by high performance liquid chromatography,” Journal of Lipid Research, 23(9):1362-1367 (1982).

In one embodiment the invention raises measurable blood NAD concentration, which is reflective of the intracellular NAD concentration of erythrocytes, or red blood cells (RBCs).

In one embodiment the NAD concentration is measured by extraction from blood samples, and measured by enzymatic spectrophotometric methods (Majamaa et al).

In some embodiments, the invention includes a method of raising RBC NAD concentration, through daily supplementation to prevent or reduce the alcohol reaction, by at least about 20 umol/L. In some embodiments, the invention includes a method of raising RBC NAD concentration, through daily supplementation to prevent or reduce the alcohol reaction, by at least about 10 umol/L. In some embodiments, the invention includes a method of raising RBC NAD concentration, through daily supplementation to prevent or reduce the alcohol reaction, by at least about 4 umol/L. Increases are measured relative to the levels of NAD prior to the start of supplementation.

In some embodiments, the invention includes a method of raising RBC NAD concentration, through daily supplementation to prevent or reduce the alcohol reaction, by at least 500%. In some embodiments, the invention includes a method of raising RBC NAD concentration, through daily supplementation to prevent or reduce the alcohol reaction, by at least about 300%. In some embodiments, the invention includes a method of raising RBC NAD concentration, through daily supplementation to prevent or reduce the alcohol reaction, by at least about 100%. In some embodiments, the invention includes a method of raising RBC NAD concentration, through daily supplementation to prevent or reduce the alcohol reaction, by at least about 50%. Increases are measured relative to the levels of NAD prior to the start of supplementation.

In some embodiments, the invention includes a method of raising blood glutathione concentration, through daily supplementation to prevent or reduce the alcohol reaction, by at least about 1 mmol/L. In some embodiments, the invention includes a method of raising blood glutathione concentration, through daily supplementation to prevent or reduce the alcohol reaction, by at least about 0.5 mmol/L. In some embodiments, the invention includes a method of raising blood glutathione concentration, through daily supplementation to prevent or reduce the alcohol reaction, by at least about 0.25 mmol/L. In some embodiments, the invention includes a method of raising blood glutathione concentration, through daily supplementation to prevent or reduce the alcohol reaction, by at least about 0.1 mmol/L. Increases are measured relative to the levels of glutathione prior to the start of supplementation.

In some embodiments, the invention includes a method of raising blood glutathione concentration, through daily supplementation to prevent or reduce the alcohol reaction, by at least about 50%. In some embodiments, the invention includes a method of raising blood glutathione concentration, through daily supplementation to prevent or reduce the alcohol reaction, by at least about 25%. In some embodiments, the invention includes a method of raising blood glutathione concentration, through daily supplementation to prevent or reduce the alcohol reaction, by at least about 10%. In some embodiments, the invention includes a method of raising blood glutathione concentration, through daily supplementation to prevent or reduce the alcohol reaction, by at least about 5%. Increases are measured relative to the levels of glutathione prior to the start of supplementation.

In some embodiments, the invention includes a method of raising blood all-trans retinoic acid concentration, through daily supplementation to prevent or reduce the alcohol reaction, by at least about 2 ng/mL. In some embodiments, the invention includes a method of raising blood all-trans retinoic acid concentration, through daily supplementation to prevent or reduce the alcohol reaction, by at least about 1 ng/mL. In some embodiments, the invention includes a method of raising blood all-trans retinoic acid concentration, through daily supplementation to prevent or reduce the alcohol reaction, by at least about 0.5 ng/mL. In some embodiments, the invention includes a method of raising blood all-trans retinoic acid concentration, through daily supplementation to prevent or reduce the alcohol reaction, by at least about 0.25 ng/mL. Increases are measured relative to the levels of all-trans retinoic acid prior to the start of supplementation.

In some embodiments, the invention includes a method of raising blood all-trans retinoic acid concentration, through daily supplementation to prevent or reduce the alcohol reaction, by at least about 75%. In some embodiments, the invention includes a method of raising blood all-trans retinoic acid concentration, through daily supplementation to prevent or reduce the alcohol reaction, by at least about 50%. In some embodiments, the invention includes a method of raising blood all-trans retinoic acid concentration, through daily supplementation to prevent or reduce the alcohol reaction, by at least about 25%. In some embodiments, the invention includes a method of raising blood all-trans retinoic acid concentration, through daily supplementation to prevent or reduce the alcohol reaction, by at least about 10%. Increases are measured relative to the levels of all-trans retinoic acid prior to the start of supplementation.

### Ingredients of the Anti-Alcohol Reaction Supplement

The combination anti-alcohol reaction supplement comprises various ingredients, which are described herein.

Agents Enhancing Intracellular or Intramitochondrial Concentration of NAD, Such as Nicotinamide:

As mutant ALDH2 has a Michaelis constant K_{M} for NAD about 150 times higher than wild-type, a much higher concentration of NAD is required for optimal enzyme activity. Thus, elevation of the concentration of NAD provides more cofactor for ALDH2, increasing the breakdown of acetaldehyde at a corresponding rate, reducing the level of accumulation.

Nicotinamide (niacinamide) is a preferred agent for enhancing intracellular and intramitochondrial NAD concentrations. Nicotinamide, or Vitamin B3, is a precursor for NAD. One study indicated that nicotinamide supplementation was responsible for raising NAD concentrations 24 fold. The amount of nicotinamide in the supplement can range from about 10 mg to about 2,500 mg, from 500 mg to about 1,500 mg, preferably about 750 mg to about 1,250 mg, more preferably about 1000 mg or about 750 mg.

If niacin is used as the agent for enhancing NAD concentrations, the amount of niacin in the supplement can range from about 10 mg to 2,500 mg, or from about 500 mg to 1,500 mg. In another embodiment the amount of niacin in the supplement is about 1000 mg. In another embodiment the amount of niacin in the supplement is about 750 mg.

If nicotinamide riboside is used as the agent for enhancing NAD concentrations, the amount of nicotinamide riboside in the supplement can range from about 10 mg to 2,500 mg, or from about 500 mg to 1,500 mg. In another embodiment the amount of nicotinamide riboside in the supplement is about 1000 mg. In another embodiment the amount of nicotinamide riboside in the supplement is about 750 mg.

If oxaloacetate is used is used as the agent for enhancing NAD concentrations, the amount of oxaloacetate in the supplement can range from about 10 mg to 1000 mg, or from about 50 mg to 250 mg. In another embodiment the amount of oxaloacetate in the supplement is about 150 mg. In another embodiment the amount of oxaloacetate in the supplement is about 200 mg.

Agents Enhancing Glutathione Concentration, Such as N-Acetyl Cysteine:

N-Acetyl Cysteine (NAC) is a preferred agent used to enhance the level of the anti-oxidant glutathione. Other agents that can be used for this purpose include Vitamin C, alpha lipoic acid, L-cysteine, glutathione, glycine, L-glutamic acid, adenosylmethionine, or conjugated linoleic acid. Cysteine is a precursor to glutathione, which can help protect against cellular damage. NAC is more bioavailable than L-cysteine itself and serves multiple purposes. Both NAC as well as glutathione contain sulfur groups that can react directly with acetaldehyde, clearing it from the cell. Glutathione is an activator of the enzyme ALDH2, increasing the breakdown of acetaldehyde, as well as an inhibitor of ADH, reducing acetaldehyde build-up. Finally, NAC is also a necessary precursor for the compound Coenzyme A. When acetaldehyde is converted to acetic acid by ALDH2, the acetic acid produced is bound to Coenzyme A and enters the Citric Acid Cycle to produce energy for the cell. Thus, enhancement of Coenzyme A levels is advantageous in clearing the acetic acid. Together with pantothenic acid, NAC can raise Coenzyme A levels and enhance the enzyme kinetics of the ALDH2 reaction. Coenzyme A is also a slight inhibitor of ADH, reducing acetaldehyde build-up.

If N-acetyl cysteine is used, the amount of N-acetyl cysteine in the supplement can range from about 10 mg to about 2,000 mg, or from about 50 mg to about 1000 mg, or from about 100 mg to about 500 mg, or from about 200 mg to about 500 mg, or from about 500 mg to about 1000 mg. In another embodiment the amount of N-Acetyl Cysteine in the supplement is about 500 mg. In another embodiment the amount of N-Acetyl Cysteine in the supplement is about 300 mg. In other embodiments, the amount of N-acetyl cysteine in the supplement can range from about 200 mg to about 600 mg, or about 300 mg to about 500 mg, or about 400 mg.

If Vitamin C is used, the amount of Vitamin C in the supplement can range from about 10 mg to about 3,000 mg, or from about 50 mg to about 1000 mg, or from about 100 mg to about 500 mg, or from about 200 mg to about 500 mg, or from about 500 mg to about 2,000 mg. In another embodiment the amount of Vitamin C in the supplement is about 1000 mg. In another embodiment the amount of Vitamin C in the supplement is about 150 mg. In another embodiment the amount of Vitamin C in the supplement is about 500 mg.

If alpha lipoic acid is used, the amount of alpha lipoic acid in the supplement can range from about 10 mg to about 500 mg, or from about 50 mg to about 300 mg, or from about 100 mg to about 300 mg. In another embodiment the amount of alpha lipoic acid in the supplement is about 150 mg. In another embodiment the amount of alpha lipoic acid in the supplement is about 250 mg.

If glutathione is used, the amount of glutathione in the supplement can range from about 10 mg to about 1,500 mg, or from about 100 mg to about 1000 mg, or from about 100 mg to about 500 mg. In another embodiment the amount of glutathione in the supplement is about 500 mg.

If L-cysteine is used, the amount of L-cysteine in the supplement can range from about 10 mg to about 2,000 mg, or from about 50 mg to about 1000 mg, or from about 100 mg to about 500 mg, or from about 200 mg to about 500 mg, or from about 500 mg to about 1000 mg. In another embodiment the amount of L-cysteine in the supplement is about 500 mg. In another embodiment the amount of L-cysteine in the supplement is about 300 mg.

If glycine is used, the amount of glycine in the supplement can range from about 10 mg to about 2000 mg, or from about 50 mg to about 1000 mg, or from about 100 mg to about 500 mg, or from about 200 mg to about 500 mg, or from about 500 mg to about 1000 mg. In another embodiment the amount of glycine in the supplement is about 500 mg. In another embodiment the amount of glycine in the supplement is about 300 mg.

If L-glutamic acid is used, the amount of L-glutamic acid in the supplement can range from about 10 mg to about 2,000 mg, or from about 50 mg to about 1000 mg, or from about 100 mg to about 500 mg, or from about 200 mg to about 500 mg, or from about 500 mg to about 1000 mg. In another embodiment the amount of L-glutamic acid in the supplement is about 500 mg. In another embodiment the amount of L-glutamic acid in the supplement is about 300 mg.

If adenosylmethionine (SAMe) is used, the amount of SAMe in the supplement can range from about 10 mg to about 500 mg, or from about 50 mg to about 250 mg. In another embodiment the amount of SAMe in the supplement is about 150 mg. In another embodiment the amount of SAMe in the supplement is about 250 mg.

If conjugated linoleic acid is used, the amount of conjugated linoleic acid in the supplement can range from about 10 mg to about 500 mg, or from about 50 mg to about 300 mg, or from about 100 mg to about 300 mg. In another embodiment the amount of conjugated linoleic acid in the supplement is about 150 mg. In another embodiment the amount of conjugated linoleic acid in the supplement is about 250 mg.

Anti-Oxidants, Such as Vitamin C:

The supplement also includes at least one anti-oxidant. Vitamin C is a well-documented anti-oxidant. Supplementation with Vitamin C can increase levels of glutathione by 50% in only two weeks. In addition, Vitamin C has general protective properties as an anti-oxidant, and is relatively safe at high doses.

The amount of Vitamin C in the supplement can range from about 100 mg to about 500 mg, preferably about 200 mg to about 400 mg, more preferably about 300 mg.

Vitamin E or other anti-oxidants can be employed instead of or in addition to Vitamin C. Alternatively, alpha-lipoic acid, another agent used in the supplement as a separate ingredient, can also be employed as an anti-oxidant.

Alpha Lipoic Acid:

Alpha Lipoic Acid is an anti-oxidant, and its use in the supplement helps to maintain high concentrations of glutathione. This occurs due to regeneration of reduced glutathione. The R-enantiomer of alpha lipoic acid is used in the supplement (5-[(3R)-dithiolan-3-yl]pentanoic acid).

The amount of alpha lipoic acid in the supplement can range from about 50 mg to about 250 mg, preferably about 100 mg to about 200 mg, more preferably about 150 mg. If alpha lipoic acid is also used as the anti-oxidant, approximately double this amount is used, that is, from about 100 mg to about 500 mg, preferably about 200 mg to about 400 mg, more preferably about 300 mg. If racemic alpha lipoic acid is used in the supplement, the foregoing amounts are doubled, so as to provide the specified amount of the R-isomer in the supplement. That is, if 300 mg of R-alpha lipoic acid is desired in the supplement, and the racemic mixture is used to make the supplement, 600 mg of the racemic mixture is used in order to provide 300 mg of the R-enantiomer.

Agents Enhancing the Level of Coenzyme A, Such as Pantothenic Acid:

As described previously, increased Coenzyme A has two functions in lowering blood acetaldehyde. Pantothenic acid is a precursor to Coenzyme A and is a preferred agent for use in the supplement to enhance the levels of Coenzyme A.

The agent capable of raising intracellular Coenzyme A concentration in the subject can be pantothenic acid, pantethine, pantetheine, 4′-phosphopantothenate, 4′-phosphopantetheine, N-acetyl cysteine, or L-cysteine.

If pantothenic acid is used (also known as pantothenate), the amount of pantothenic acid in the supplement can range from about 10 mg to 1000 mg, or from about 100 mg to 500 mg, or from about 50 mg to about 250 mg, or from about 100 mg to about 200 mg, or about 150 mg. In another embodiment the amount of pantothenic acid in the supplement is about 50 mg. In another embodiment the amount of pantothenic acid in the supplement is about 250 mg.

If pantethine is used, the amount of pantethine in the supplement can range from about 10 mg to about 1000 mg, or from about 100 mg to about 500 mg. In another embodiment the amount of pantethine in the supplement is about 50 mg. In another embodiment the amount of pantethine in the supplement is about 250 mg.

If 4′-phosphopantetheine is used, the amount of 4′-phosphopantetheine in the supplement can range from about 10 mg to about 1000 mg, or from about 100 mg to about 500 mg. In another embodiment the amount of 4′-phosphopantetheine in the supplement is about 50 mg. In another embodiment the amount of 4′-phosphopantetheine in the supplement is about 250 mg.

If 4′-phosphopantothenate is used, the amount of 4′-phosphopantothenate in the supplement can range from about 10 mg to about 1000 mg, or from about 100 mg to about 500 mg. In another embodiment the amount of 4′-phosphopantothenate in the supplement is about 50 mg. In another embodiment the amount of 4′-phosphopantothenate in the supplement is about 250 mg.

If N-acetyl cysteine is used as the agent capable of raising intracellular Coenzyme A concentration, the amount of N-acetyl cysteine used in the supplement for this purpose can range from about 10 mg to about 2,000 mg, or from about 50 mg to about 1000 mg, or from about 100 mg to about 500 mg, or from about 200 mg to about 500 mg, or from about 500 mg to about 1000 mg. In another embodiment the amount of N-acetyl cysteine in the supplement is about 500 mg. In another embodiment the amount of N-acetyl cysteine in the supplement is about 300 mg. The amount of N-acetylcysteine added for use as the agent capable of raising intracellular Coenzyme A concentration is in addition to the amount used if N-acetylcysteine is used as the agent capable of increasing glutathione concentration.

If L-cysteine is used as the agent capable of raising intracellular Coenzyme A concentration, the amount of L-cysteine used in the supplement for this purpose can range from about 10 mg to about 2,000 mg, or from about 50 mg to about 1000 mg, or from about 100 mg to about 500 mg, or from about 200 mg to about 500 mg, or from about 500 mg to about 1000 mg. In another embodiment the amount of L-cysteine in the supplement is about 500 mg. In another embodiment the amount of L-cysteine in the supplement is about 300 mg. The amount of L-cysteine added for use as the agent capable of raising intracellular Coenzyme A concentration is in addition to the amount used if L-cysteine is used as the agent capable of increasing glutathione concentration.

Agents Enhancing the Level of All-Trans Retinoic Acid:

beta-carotene or a retinol ester can be used to raise concentrations of all-trans retinoic acid in a subject. Examples of esters of retinol that can be used are retinyl arachidonate, retinyl stearate, retinyl oleate, retinyl linoleate, retinyl heptadecanoate, retinyl palmitate, retinyl myristate, retinyl laurate, retinyl caprylate, or retinyl acetate.

If beta-carotene is used, the amount of beta-carotene in the supplement can range from about 100 IU to about 15,000 IU, or from about 1000 IU to about 10,000 IU. In another embodiment the amount of beta-carotene in the supplement is about 2,500 IU. In another embodiment the amount of beta-carotene in the supplement is about 5000 IU.

If a retinol ester is used, the amount of retinol ester in the supplement can range from about 100 IU to about 15,000 IU, or from about 1000 IU to about 10,000 IU. In another embodiment the amount of retinol ester in the supplement is about 2500 IU. In another embodiment the amount of retinol ester in the supplement is about 5000 IU.

Silybum marianum Extract.

Silybum marianum (milk thistle) is an annual or biannual plant of the Asteraceae family. It has been used as a medicinal plant for centuries, and its extract is used in medicine under the name silymarin. Milk thistle is typically extracted with 95% ethanol, which may then be filtered and dried by solvent evaporation. The resulting extract typically contains 1.5-3% flavonolignans (referred to collectively as silymarin; the principal components are silybin and isosilybin), 20-30% fatty acids (including linoleic, oleic, and palmitic acids), 25-30% proteins, and small amounts of vitamin E and various sterols. It may be included in the any of the compositions of the present invention as disclosed herein at concentrations between 100-5000 mg/day. Additional information regarding this extract is available from the World Health Organization at URL apps.who.int/medicinedocs/pdf/s4927e/s4927e.pdf, pages 300-316.

Hovenia dulcis Fruit Extract.

Hovenia dulcis (oriental raisin tree) is a tree found in Asia, over Eastern China, Korea, and in the Himalayan mountains (found at altitudes up to 2,000 m). There is evidence that extracts of the Hovenia dulcis fruit are hepatoprotective and can reduce blood alcohol levels (see Hyun et al., Planta Medica 76:943-949 (2010) and references therein). The main chemical for this effect in Hovenia dulcis is quercetin, which is believed to act as an anti-inflammatory and anti-oxidant. Another component of Hovenia dulcis, ampelopsin (dihydromyricetin), also affects alcohol metabolism and is discussed in Shen et al., The Journal of Neuroscience, 32(1): 390-401 (2012). For treating a human patient, a typical daily dose of the above-mentioned extract may range from about 0.01 to 10 g/kg body weight, preferably 1 to 5 g/kg body weight. A lower alcohol-insoluble fraction and the polysaccharide isolated therefrom can be used in tablet or capsule form, or may be added to food or beverage. The amount of the fraction and/or polysaccharide may generally range from about 0.1 to 15 w/w %, preferably 1 to 10 w/w % based on the total weight of food, and 0.1 to 15 g, preferably 1 to 10 g.

### Dosing/Administration

The compositions of the invention comprising the supplement can be administered in various regimens. The compositions can be administered to a subject prior to consumption of alcohol by the subject. In some embodiments, the compositions are administered to the subject for at least three days prior to consumption of alcohol by the subject, such as periods of at least about seven days, at least about 14 days, at least about a month, or at least about three months.

The compositions can be administered in a single daily dose, where the dose contains the entire amount of the composition to be administered on that day. Alternatively, the compositions can be administered in multiple divided doses over the course of one day, such that the multiple doses administered add up to the entire amount of the composition to be administered on that day. Thus, one-half of the daily dosage can be administered twice a day, one-third of the daily dosage can be administered three times a day, one-fourth of the daily dosage can be administered four times a day, one-fifth of the daily dosage can be administered five times a day, etcetera. In yet another embodiment, the individual components of the supplement can be administered separately, or in any sub-combination, over the course of the day; for example, the amount of anti-oxidant and the amount of alpha-lipoic acid can be administered in the morning, and the amount of the agents capable of increasing intracellular or intramitochondrial concentration of NAD, of increasing glutathione concentration, and of increasing Coenzyme A can be administered in the evening.

### Formulations and Routes of Administration

For the purposes of this disclosure, the compounds may be administered by a variety of means including orally, parenterally, by inhalation spray, topically, or rectally in formulations containing pharmaceutically acceptable carriers, adjuvants and vehicles. The term oral as used herein includes, but is not limited to, sublingual and buccal. Oral administration includes fluid drinks, energy bars, as well as pill formulations. Oral administration by swallowing the compounds is advantageous due to its convenience. However, a patient who has consumed a great deal of alcohol may have difficulty in swallowing, or may be unconscious, in which case introduction of the supplements by other means (feeding tube, intravenously, etc.) may be desirable.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous-suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an anti-oxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

As noted above, formulations of the disclosure suitable for oral administration may be presented as discrete units such as capsules, cachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide. slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of formula I when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

As used herein, pharmaceutically acceptable salts include, but are not limited to: acetate, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Additional pharmaceutically acceptable salts are known to those skilled in the art.

The compositions of the present invention may also be formulated as nutraceutical compositions. The term “nutraceutical composition” as used herein refers to a food product, foodstuff, dietary supplement, nutritional supplement or a supplement composition for a food product or a foodstuff comprising the compositions of the present invention added exogenously. As used herein, the term food product refers to any food or feed suitable for consumption by humans or animals. The food product may be a prepared and packaged food (e.g., mayonnaise, salad dressing, bread, grain bar, beverage, etc.) or an animal feed (e.g., extruded and pelleted animal feed, coarse mixed feed or pet food composition). As used herein, the term foodstuff refers to any substance fit for human or animal consumption.

Food products or foodstuffs are for example beverages such as non-alcoholic drinks as well as liquid preparation to be added to drinking water and liquid food, non-alcoholic drinks are for instance soft drinks, sport drinks, fruit juices, such as for example orange juice, apple juice and grapefruit juice; lemonades, teas, near-water drinks and milk and other dairy drinks such as for example yoghurt drinks, and diet drinks. In another embodiment food products or foodstuffs refer to solid or semi-solid foods comprising the composition according to the invention. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

Animal feed including pet food compositions advantageously include food intended to supply necessary dietary requirements, as well as treats (e.g., dog biscuits) or other food supplements. The animal feed comprising the composition according to the invention may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

The term dietary supplement refers to a small amount of a compound for supplementation of a human or animal diet packaged in single or multiple dose units. Dietary supplements do not generally provide significant amounts of calories but may contain other micronutrients (e.g., vitamins or minerals). The term food products or foodstuffs also includes functional foods and prepared food products pre-packaged for human consumption.

The term nutritional supplement refers to a composition comprising a dietary supplement in combination with a source of calories. In some embodiments, nutritional supplements are meal replacements or supplements (e.g., nutrient or energy bars or nutrient beverages or concentrates).

Dietary supplements of the present invention may be delivered in any suitable format. In preferred embodiments, dietary supplements are formulated for oral delivery. The ingredients of the dietary supplement of this invention are contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a hard (shell) capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate.

In other embodiments, the dietary supplement is provided as a powder or liquid suitable for adding by the consumer to a food or beverage. For example, in some embodiments, the dietary supplement can be administered to an individual in the form of a powder, for instance to be used by mixing into a beverage, or by stifling into a semi-solid food such as a pudding, topping, sauce, puree, cooked cereal, or salad dressing, for instance, or by otherwise adding to a food or the dietary supplement e.g. enclosed in caps of food or beverage container for release immediately before consumption. The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like.

In some embodiments, the dietary supplements further comprise additional vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; zinc sulfate or oxide; copper gluconate; riboflavin; beta-carotene; pyridoxine hydrochloride; thiamin mononitrate; folic acid; biotin; chromium chloride or picolinate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines and the disclosure herein.

In other embodiments, the present invention provides nutritional supplements (e.g., energy bars or meal replacement bars or beverages) comprising the composition according to the invention. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and hydrolysates or mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modem Nutrition in Health and Disease, 8^{th} ed., Lea & Febiger, 1986, especially Volume 1, pages 30-32. The nutritional supplement can also contain other ingredients, such as one or a combination of other vitamins, minerals, anti-oxidants, fiber and other dietary supplements (e.g., protein, amino acids, choline, lecithin). Selection of one or several of these ingredients is a matter of formulation, design, consumer preferences and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolinate; potassium iodide; sodium selenate; sodium molybdate; phylloquinone; vitamin D3; cyanocobalamin; sodium selenite; copper sulfate; vitamin A; vitamin C; inositol; potassium iodide.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

It is understood by those of skill in the art that other ingredients can be added to those described herein, for example, fillers, emulsifiers, preservatives, etc. for the processing or manufacture of a nutritional supplement.

Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

In addition to the preparations described above, standard pharmaceutical preparations, such as those described in Remington: The Science and Practice of Pharmacy (22^{nd} Edition, Pharmaceutical Press, 2012 and 21^{st} Edition, Pharmaceutical Press, 2011), can be used.

### Kits

The invention also provides articles of manufacture and kits containing the compositions described herein. The article of manufacture comprises a container with a label. Suitable containers include, for example, bottles, vials, and test tubes. The containers may be formed from a variety of materials such as glass or plastic. The containers can hold one or more of the individual ingredients. The label on the container indicates that the kit is used for prevention or reduction of one or more symptoms associated with the alcohol reaction.

### EXAMPLES

### Example 1

### Supplement Formulations

The supplement can be prepared with the following amounts of individual components:

### Formulation A:

### Nicotinamide: 1000 mg

### N Acetyl Cysteine: 400 mg

### Vitamin C: 300 mg

### Alpha-Lipoic Acid: 150 mg

### Pantothenic Acid: 150 mg

### Formulation B:

### Nicotinamide: 800 mg

### N Acetyl Cysteine: 300 mg

### Vitamin C: 150 mg

### Alpha-Lipoic Acid: 100 mg

### Pantothenic Acid: 100 mg

### Formulation C:

### Nicotinamide: 700 mg

### N Acetyl Cysteine: 300 mg

### Vitamin C: 150 mg

### Pantothenic Acid: 100 mg

### Vitamin A: 2500 IU

### Thiamine: 50 mg

### Formulation D:

### N Acetyl Cysteine: 300 mg

### Nicotinamide: 700 mg

### Pantothenic Acid: 100 mg

### Example 2

### Demonstration of Efficacy of Combination Anti-Alcohol Reaction Supplement Therapy

The efficacy of the combination anti-alcohol reaction supplement (hereafter “supplement”) is demonstrated in the following manner. A double blind supplement trial is conducted where subjects take the supplement (or placebo) once daily for three weeks. Either Formulation A or Formulation B of Example 1 is used with all subjects. Subjects provide informed consent for participation in accordance with all laws and regulations, and all policies of the institution where the study is conducted. The age of subjects is verified to ensure that they are of legal age to consume alcohol in the jurisdiction where the study takes place (typically, age 21 years or older in the United States). Subjects that qualify under the inclusion criteria of the study come in for a first visit, where their health and ability to participate in the study are assessed by the staff. At this initial visit, subjects first have their blood acetaldehyde level measured before drinking any alcohol. Subjects then consume two standard alcoholic drinks. Three more blood tests for acetaldehyde after alcoholic beverage consumption are conducted at time points of 15 minutes, 45 minutes and 90 minutes after finishing the drinks. Each patient must have suitable transportation to and from the research facility in order to avoid driving while under the influence of alcohol. Patient privacy and confidentiality is maintained according to law and regulation. Patients can also fill out questionnaires indicating the degree to which they experience facial flushing, dizziness, nausea, headache, stupor, or increased heart rate at the same time points at which the blood tests are conducted. The degree of facial flushing can also be reported by the staff (either by visual observation or by measurement with a chromameter), and heart rate can be monitored.

Subjects commence taking supplement (or placebo) daily for three weeks, starting the day after the initial visit. The double-blind trial utilizes three groups: one group consisting of individuals with ALDH2 deficiency who take the supplement, one group of individuals without the deficiency (that is, they do not demonstrate the alcohol reaction) who take the supplement, and a third group of individuals who do have ALDH2 deficiency, but who take a placebo.

The subjects visit the research facility again three weeks later. The same steps are performed as were conducted during the initial visit of the subjects. Again, subjects have a blood test to determine acetaldehyde levels, consume two alcoholic drinks, and have three more blood tests at time points of 15 minutes, 45 minutes and 90 minutes after finishing the drinks in order to measure the levels of acetaldehyde in the blood. Patients can also fill out questionnaires indicating the degree to which they experience facial flushing, dizziness, nausea, or increased heart rate at the same time points at which the blood tests are conducted. The degree of facial flushing can also be reported by the staff (using the same method—either by visual observation or by measurement with a chromameter—as used previously), and heart rate can be monitored. The data is compared between visits one and two to demonstrate the effects of the supplement in preventing accumulation of acetaldehyde.

### Example 3

### Demonstration of Efficacy of Combination Anti-Alcohol Reaction Supplement Therapy

The efficacy of the combination anti-alcohol reaction supplement (hereafter “supplement”) is demonstrated in the following manner. A double blind supplement trial is conducted where subjects take the supplement (or placebo) once daily for 30 days. One group will not supplement for 30 days and will take a single dose prior to consuming alcohol. Either Formulation C or Formulation D of Example 1 is used with all subjects. Subjects provide informed consent for participation in accordance with all laws and regulations, and all policies of the institution where the study is conducted. The age of subjects is verified to ensure that they are of legal age to consume alcohol in the jurisdiction where the study takes place (typically, age 21 years or older in the United States). Subjects that qualify under the inclusion criteria of the study come in for a first visit, where their health and ability to participate in the study are assessed by the staff. At this initial visit there will be samples taken for a PCR genotyping to confirm that all subjects with self-reported facial flushing when drinking, are heterozygous for the ALDH2 mutation. If they do possess this mutation and pass the inclusion criteria, they will visit the facility for a 2^{nd} visit. At this 2^{nd} visit, subjects first have their blood acetaldehyde level measured before drinking any alcohol. Subjects then consume 0.3 g/Kg body weight of ethanol. Three more blood tests for acetaldehyde after ethanol consumption are conducted immediately after drinking, and at two more time points, each with 20 minutes of separation. At each of these time points there will be measurements for heart rate and skin temperature. Each patient must have suitable transportation to and from the research facility in order to avoid driving while under the influence of alcohol. Patient privacy and confidentiality is maintained according to law and regulation. Patients can also fill out questionnaires indicating the degree to which they experience facial flushing, dizziness, nausea, headache, stupor, or increased heart rate at the same time points at which the blood tests are conducted. The degree of facial flushing can also be reported by the staff (either by visual observation or by measurement with a chromameter), as well as self-reported by the subject.

Subjects commence taking supplement (or placebo) daily for 30 days, starting the day after the initial visit. The double-blind trial utilizes 4 groups: one group consisting of individuals with ALDH2 deficiency who take the supplement for 30 days (formulation C), one group of individuals with the ALDH2 deficiency who take a subset of the supplement for 30 days (formulation D), one group of individuals with the ALDH2 deficiency, but who take a placebo for 30 days, and one group of individuals with the ALDH2 deficiency who do NOT supplement for 30 days, but take a single dose of the supplement (formulation C) at the third visit.

The subjects visit the research facility again for a 3^{rd} visit 30 days later. The same steps are performed as were conducted during the 2^{nd} visit, with one addition. Again, subjects have a blood test to determine acetaldehyde levels. All subjects (except placebo) will then consume one dosage, 20 minutes prior to consuming alcohol. Subjects will then consume 0.3 g/Kg body weight of ethanol. Three more blood tests for acetaldehyde after ethanol consumption are conducted immediately after drinking, and at two more time points, each with 20 minutes of separation. At each of these time points there will be measurements for heart rate and skin temperature. Patients can also fill out questionnaires indicating the degree to which they experience facial flushing, dizziness, nausea, or increased heart rate at the same time points at which the blood tests are conducted. The degree of facial flushing can also be reported by the staff (using the same method—either by visual observation or by measurement with a chromameter—as used previously), and can also be self-reported by the subject. The data is compared between visits two and three to demonstrate the effects of the supplement in preventing accumulation of acetaldehyde, relevant to the placebo group.

### Example 4

### Additional Demonstration of Efficacy of Combination Anti-Alcohol Reaction Supplement Therapy

The efficacy of the combination anti-alcohol reaction supplement (hereafter “supplement”) can also be demonstrated in a double blind trial, where subjects take the supplement (or placebo) twice daily for three weeks. Formulation A, Formulation B, Formulation C, or Formulation D of Example 1 is used with all subjects. Other than the twice-daily administration of supplement (or placebo), the trial is carried out as described in Example 2.

### Example 5

### Studies in Individuals of Efficacy of Combination Anti-Alcohol Reaction Supplement Therapy

In order to evaluate the efficacy of certain embodiments of the invention, subjects took various forms of the supplements daily for two weeks. The subjects then consumed two standard alcohol beverages and their symptoms were evaluated. One hour prior to drinking the alcohol beverages, the subjects also consumed another dose of the formulation. Overt physical symptoms and self-reported physical symptoms were used as assessments for the alcohol reaction. These experimental tests include eleven subjects. All experimental subjects were unpaid volunteers who experience all symptoms of the alcohol reaction.

The following symptoms were assessed: facial redness, heart rate, self-evaluated body temperature, headache, nausea, and general physical discomfort. These are symptoms of the alcohol reaction that have all been directly linked with an increase in blood acetaldehyde.

Subject 1 (male) took a supplement containing 500 mg of N-Acetyl Cysteine, 500 mg Vitamin C and 1000 mg nicotinamide. After the supplementation period, upon consumption of alcohol, subject 1 experienced immediately noticeable reduction in facial redness, headache, and body temperature from what the subject typically previously experienced in response to the same amount of alcohol consumption.

Subject 2 (female) and Subject 3 (male) took a supplement containing 500 mg of N-Acetyl Cysteine, 100 mg pantothenic acid, 500 mg Vitamin C, and 1000 mg nicotinamide. Upon consumption of alcohol, Subject 2 displayed a significant reduction in both physical discomfort and headache. Subject 3 displayed mild reductions in all assessments for the alcohol reaction.

Subject 4 (male) took a supplement containing only with 1500 mg nicotinamide. The subject reported reductions in facial redness and heart rate.

Subject 5 (female), Subject 6 (female), and Subject 7 (male) took a supplement containing 500 mg N-Acetyl Cysteine, 100 mg Pantothenic acid, 500 mg Vitamin C, 1000 mg nicotinamide and 150 mg Alpha Lipoic Acid. Subject 5 displayed mild reductions in all alcohol reaction assessments, with a significant reduction in heart rate. Subject 6 displayed a significant reduction in heart rate and headache, and a reduction in physical discomfort and facial flushing. Subject 7 displayed dramatic decreases in heart rate, nausea, physical discomfort, and body temperature, and a mild reduction in facial redness.

Subject 8 (female) took a supplement containing 2500 IU Vitamin A, 700 mg Nicotinamide, 25 mg pantothenic acid, 150 mg Vitamin C, 125 mg Alpha Lipoic Acid, 300 mg N-Acetyl Cysteine, and 10 mg Thiamine Subject 8 displayed dramatic reductions in all assessments for the alcohol reaction, with zero displayed facial redness.

Subject 9 (female), Subject 10 (female), and Subject 11 (male) took a supplement containing 2500 IU Vitamin A, 700 mg Nicotinamide, 50 mg Pantothenic acid, 150 mg Vitamin C, 80 mg Alpha Lipoic Acid, 300 mg N-Acetyl Cysteine, and 50 mg Thiamine. Subject 11 did not display reductions in any assessments. Subject 9 displayed a dramatic reduction in heart rate, and mild reductions in facial redness, body temperature, headache, nausea, and physical discomfort. Subject 10 displayed significant reductions in all assessments.

Different individuals display varying degrees of severity for the symptoms of the alcohol reaction. This suggests that at a given concentration of acetaldehyde in the blood, individuals experience varying intensities of the alcohol reaction symptoms assessed in these experiments. This is confirmed by the experimental results, in that 10 out of 11 individuals displayed reductions in at least one symptom, although the symptoms varied. The results suggest that various embodiments of the invention reduce acetaldehyde in the blood, and individuals respond differently to that reduction. The most consistently reduced symptom was heart rate.

### Example 6

### Clinical Study Results of Anti-Alcohol Reaction Supplement Therapy

A clinical study was conducted using two different formulations of the anti-alcohol reaction supplement, along with a placebo group as a control group.

Study Products and Doses:

Table A lists the active components for the two formulations to be tested in this study: NoGlo™ and the formula for NoGlo-2™.

TABLE A
Study Products (per 2 capsules)

| | NoGlo ™ | NoGlo-2 ™ Supplement |
|---|---|---|
| | Concentration | Concentration |
| Component | (mg) | (mg) |
| Nicotinamide | 700 | 700 |
| N-Acetyl-Cysteine | 300 | 300 |
| Pantothenic Acid | 50 | 50 |
| Vitamin A* | 2500 IU (1.5 mg) | |
| Thiamine | 50 | |
| Vitamin C | 150 | |
| Alpha Lipoic Acid | 80 | |
| Total Weight | 1331.5 (approx..) | 1050 |

*as beta-carotene; 2500 IU Vitamin A as beta-carotene = 1.5 mg

The inactive ingredients for NoGlo™ are as follows: Silicone Dioxide, 8 mg; Magnesium Stearate, 20 mg. The product is in Size 1 blue opaque gelatin capsules. The inactive ingredients for NoGlo-2™ are as follows: Rice Flour, 100 mg; Silicone Dioxide, 8 mg; Magnesium Stearate, 20 mg; the product is in Size 1 blue opaque gelatin capsules. Placebo will be identical to NoGlo™ but without the active ingredients.

Alcohol:

The alcohol product for use in the study is Absolut® Vodka (40% alcohol/80 proof) in 280 ml (9.5 oz.) of Tropicana® Pure Premium Orange juice. (ABSOLUT is a registered trademark of The Absolut Company, Stockholm, Sweden. TROPICANA is a registered trademark of Tropicana Products, Inc., East Bradenton, Fla., United States.)

Product Administration/Study Products:

This is a 20 subject, 3 group study. The groups will be administered treatments as follows:

Group 1 (n=12): At baseline placebo 0.5 hr before alcohol administration followed by 28 days of NoGlo. On day 28 an additional dose of NoGlo 0.5 hr before alcohol administration.

Group 2 (n=4): At baseline placebo 0.5 hr before alcohol administration followed by 28 days of NoGlo-2. On day 28 an additional dose of NoGlo-2, 0.5 hr before alcohol administration.

Group 3 (n=4): Cross over design. At baseline 2 subjects receive placebo and 2 NoGlo 0.5 hr before alcohol administration followed by 28 days of placebo. On day 28 the order for the subjects will be reversed (2 subjects receiving placebo will receive active and vice versa), 0.5 hr before alcohol administration.

Each patient will be instructed to take 4 capsules each day. The capsules should be taken with at least 150 ml (5 oz.) of water. The dose should be taken in the morning after an overnight fast, at least 30 minutes before breakfast. On baseline and day 28, at the site, subjects will take an additional dose 60 min before alcohol administration and therefore should stay fasted till the end of the observation period. A light meal will be offered after that.

Subjects will receive 0.625 ml vodka (0.25 ml of 100% ethanol) per kg of body weight in 280 ml (9.5 Oz) of orange juice at baseline and on day 28, at time 0 (see table of events lb). For example, for a 70 kg person, the dosing calculation will be as follows: 70 kg×0.625 g/Kg=43.8 g; for the purposes of the study one g is equal to one ml; one kg=2.2 pounds. The calculation for a subject weighing 170 pounds, in order to arrive at the volume in ml of vodka (40% alcohol/80 proof) to be added to the orange juice is as follows: 170 pounds×0.284 mg/pound=48.28 ml. The number of ml will be rounded up or down to the nearest ml; in this case, rounded to 48 ml. Subjects will be expected to consume the drink within 5 minutes.

The product will be manufactured by Nutritional Supplement Manufacturers, 1055 Bay Boulevard, #A, Chula Vista, Calif. 91911. They are an FDA-approved manufacturing facility and adhere to current Good Manufacturing Practices (GMP).

TABLE B
PARAMETERS (EFFICACY AND SAFETY
MEASUREMENTS) TO BE STUDIED

| | TIME OF MEASUREMENT |
|---|---|
| EFFICACY MEASURE | |
| Acetaldehyde blood levels | (Baseline and Day 28) |
| Blood pressure | (Baseline and Day 28) |
| Heart Rate- | (Baseline and Day 28) |
| Skin Temperature-both Cheeks | (Baseline and Day 28) |
| (Exergen model skin thermometer) | |
| Flushing-as determined by patient (1 | (Baseline and Day 28) |
| to 10 scale) | |
| Flushing as determined by physician | (Baseline and Day 28) |
| (1 to 10 scale) | |
| Subject questionnaire | (Baseline and Day 28) |
| SAFETY MEASURE | |
| Blood pressure | (Baseline and Day 28) |
| Heart Rate | (Baseline and Day 28) |
| Monitoring of adverse events | (Days 1-28) |
| Complete metabolic panel | (Baseline-28) |

Subject Selection Criteria:

Inclusion Criteria are: 1) healthy adult volunteers of either gender who are 21-55 years of age; 2) cooperative, able to read, understand and give informed consent, and able to adhere to the study visit schedule and protocol requirements; 3) have body weight greater than 100 pounds with a calculated Body Mass Index (BMI) greater than 19 at study entry; 4) altered alcohol metabolism due to genetic polymorphisms with known symptoms of acetaldehyde toxicity (by ALDH2*2 allele test); 5) not naïve to alcohol consumption and have consumed alcohol previously; 6) willing to consume 0.25 g of ethanol per Kg body weight (2 standard drinks for a 70 Kg individual), during two separate study visits as part of the study; 7) agree to not operate any motor vehicles after the consumption of alcohol, for at least 4 hours; 8) self-reported to be of at least 50% Chinese, Japanese, or Korean ethnicity; 9) if a woman of childbearing potential, have a negative serum pregnancy test at screening and be willing to follow adequate contraceptive measures during the study; and 10) history of flushing, and/or palpitations, and/or nausea following occasional or inadvertent ethanol consumption either currently or in the past.

Exclusion Criteria are: 1) have a personal history of alcohol abuse; 2) have taken any medications the day of study visits 2 & 3 known to interact with alcohol metabolism; 3) have allergies to any of the ingredients in the NoGlo supplement or in the alcohol combination; 4) be homozygous for the ALDH2 mutation (i.e., have two mutated alleles); 5) have been in a clinical trial within the last 1 month from screening; 6) be diabetic; 7) have a known clinically-significant concurrent medical disease; 8) have clinically significant abnormality on physical examination suggestive of a significant medical condition; or 9) be currently nursing or pregnant or suspected of being pregnant, or be unwilling to follow adequate contraceptive measures during the study.

Study Design:

Screening The PI or Clinical Research Coordinator (CRC) will prescreen potential candidates by telephone. The PI or the CRC will instruct qualified patients to come to the study site as described in the selection criteria. At the first visit (screening) to the study site, the candidate will review the Informed Consent and Bill of Rights and sign, if agreeable. PI or CRC will review with the subjects the Consent Form or Bill of Rights and what will be required of them in order to complete the study. Financial compensation will be discussed. The candidate will be given ample time to review the consent. The PI or the CRC will ask the candidate to complete a screening health questionnaire. If qualified based on all the inclusion criteria, a genetic test for ALDH2*2 allele will be carried out, which will involve having a blood spot sample collected from the fingertip for genotyping (Visit 1). This is done by making a small finger prick with a lancet and spotting blood drops onto a piece of paper.

Randomization of the Subjects: There will be 12, 4 and 4 subjects in each of the 3 groups, respectively. SAS (SAS Institute, Cary N.C.) will be used to write a program that generates a list of random treatment assignments to the various groups.

Study Protocol Description:

This study is designed as a single-center, randomized study of the effects of NoGlo™ Supplement on alcohol metabolism and blood acetaldehyde levels in individuals who have acetaldehyde dehydrogenase 2 (ALDH2) deficiencies. Twenty subjects will be randomized into 3 groups: 1. NoGlo™ supplement for 28 days, 2. NoGlo™-2 supplement for 28 days and 3. Placebo for 28 days. Subjects meeting the study inclusion/exclusion criteria, including a positive screen for the ALDH2 mutation, will receive one of the two products or placebo daily for 28 days. These products will be distributed to subjects on Visit 2 (see also product administration section for more details on dosing and below). There will be three visits to the site (see Schedule of events, Table 1a and Table 1b).

Study Visit 1 events are described above under “Screening”.

Study Visit 2 is the Baseline day. During this visit, baseline blood for acetaldehyde levels will be drawn (about 1 to 2 teaspoons per draw). Sixty minutes prior to alcohol consumption, subjects in group 1 and 2 will receive placebo in order to mimic the protocol for day 28, in the presence of the study staff. Two out of the four subjects in group 3 will receive active (NoGlo) and two will receive placebo. Group 3 will be in a cross over design and subjects will be reversed on Day 28.

Subsequently, subjects will consume 0.25 g of ethanol per kilogram of body weight. Additional blood samples for determination of acetaldehyde will be obtained at 10, 20 and 40 minutes following completion of alcohol consumption. At each of these time points, vital sign, skin temperature of both cheeks, flushing and questionnaire measurements will also be made. A breathalyzer test will be administered prior to alcohol intake and at two and four hours following alcohol. Subjects will not be released from the site until the breathalyzer reads zero or the four hour time point arrives.

Following completion of the observation period after the initial dose during Study Visit 2, subjects will receive enough study supplements for 28 days. During the dosing phase, subjects will take 4 capsules of the appropriate study supplement each morning until they return (28 days) for study visit 3.

Study Visit 3 will occur on study day 28. The subject will have taken a dose of the study supplements or placebo the morning of the 3^{rd} visit. During this visit, baseline acetaldehyde levels will be drawn (about 1 to 2 teaspoons per draw). Sixty minutes prior to alcohol consumption, subjects in group 1 and 2 will take an additional dose of the study supplements, in the presence of the study staff, according to their study group. Two out of the four subjects in group 3 will receive active (NoGlo) and two will receive placebo, in reverse order from baseline. Subsequently, subjects will consume 0.25 g of ethanol per kilogram of body weight. Additional blood samples for determination of acetaldehyde will be obtained at 10, 20 and 40 minutes following completion of alcohol consumption. At each of these time points, vital sign, skin temperature of both cheeks, flushing and questionnaire measurements will also be made. A breathalyzer test will be administered prior to alcohol intake and at two and four hours following alcohol. Subjects will not be released from the site until the breathalyzer reads zero or four hour time point reached.

It is possible that some subjects would be unable to comply with at the exact timing of the 28 days for the third site visit. In such eventuality subjects will be considered compliant if they fulfill their obligation of this visit by day 30 and that will allow them to be included in the statistical analysis. Under those conditions subjects will be required to continue taking their daily dose of supplement till their 3^{rd} visit to the site.

The primary efficacy endpoint is the change in blood acetaldehyde concentration following alcohol ingestion after 28-days' administration of study supplements compared to the concentration following alcohol ingestion at Baseline. Secondary endpoints include: Changes in heart rate and skin temperature of both cheeks following alcohol consumption comparing Day 28 to Baseline and changes in symptomatic responses following alcohol consumption comparing Day 28 to Baseline.

Statistics:

Means and standard deviations (SD) and standard errors of the mean (S.E.M) will be reported for continuous variables and percentages will be reported for categorical variables. Independent sample t-tests will be used to compare differences across groups for continuous variables. Fisher's exact tests will be used to compare categorical variables. The accepted level of significance for all tests will be a=0.05. All analyses will be performed using SAS (SAS Institute, Cary, N.C.)

Compliance:

Compliance will be enforced and monitored using the following methods: proper subject selection; subject education, management, and encouragement; telephone calls; and study supplement accountability.

TABLE 1A
SCHEDULE OF EVENTS

| | | | Day | Day 28 |
|---|---|---|---|---|
| Event | Screening | Baseline | 1-27 | (±2) |
| Informed Consent | Y | N | N | N |
| Health history | Y | N | N | N |
| Physical exam | N | Y | N | N |
| Comp Metabolic Panel | N | Y | N | Y |
| ALDH2 Deficiency test | Y | N | N | N |
| (genotype) | | | | |
| Flushing Subject & PI | N | Y | N | Y |
| (1-10 Scale) | | | | |
| Subject questionnaire | N | Y | N | Y |
| HR and BP | Y | Y | N | Y |
| Acetaldehyde in blood | N | Y | N | Y |
| Skin temperature | N | Y | N | Y |
| Administer products | N | Y | Y | Y |
| Administer alcohol | N | Y | N | Y |
| Adverse event monitoring | N | Y | Y | Y |
| Site visits | Y | Y | N | Y |

TABLE 1B
Baseline and DAY 28
Visits 2 & 3

| | (min) | | | | | | |
|---|---|---|---|---|---|---|---|
| Event | −60 | 0 | 10 | 20 | 40 | 120 | 240 |
| HR and BP | | x | x | x | x | | |
| Skin temperature | | x | x | x | x | | |
| Flushing Subject & PI (1-10 | | x | x | x | x | | |
| Scale) | | | | | | | |
| Acetaldehyde in blood | | x | x | x | x | | |
| Additional product dose or | x* | | | | | | |
| placebo | | | | | | | |
| Ethanol | | x | | | | | |
| Breathalyzer** | | x | | | | x | x |

*Subjects will fast overnight as on every day of dosing and stay fasted on baseline and day 28 till the end of the observation period.
**For informational purposes only-to release subjects from site at Baseline and day 28

### Study Results

The double blind, internal-placebo controlled study was completed on Sep. 25, 2013. The study demonstrated the positive effects of the NoGlo product. Described in the following paragraphs are the results of the measurements and tests conducted in the study relating to the cause and symptoms of the alcohol reaction (Alcohol Flush Reaction or AFR). All individuals who participated in the study were positively genotyped to be heterozygous for the ALDH2 mutation.

### Blood Acetaldehyde Concentration Over Time:

Large accumulations of blood acetaldehyde is recognized and well established as the cause of the symptoms of the alcohol reaction. An inability to breakdown acetaldehyde properly leads to its accumulation in those with the ALDH2 mutation. The aldehyde compound is 30 times as toxic as ethanol and can cause DNA and protein damage by reacting with nucleic and amino acids. In the study, acetaldehyde concentrations were measured in the blood at baseline (time 0 minutes) and then at 10, 20 and 40 minutes following the consumption of 0.25 g of ethanol per Kg body weight. The exact same tests were done before (Day 1), and after supplementation (Day 28).

Group 1 supplemented with the full NoGlo formulation as listed in the protocol. Group 2 supplemented with the NoGlo-2 supplement as listed in the protocol. Group 3 supplemented with a placebo, followed by a single dose of the full NoGlo formulation.

The following (Table 2) results are represented in the units mg/dL for blood acetaldehyde.

TABLE 2
Blood acetaldehyde over time.

| Acetaldehyde | 0 min. | 10 min. | 20 min. | 40 min. |
|---|---|---|---|---|
| | Group 1 | | | |
| Day 1 | 0.0 (0.0) | 0.76 (0.17) | 0.91 (0.19) | 0.92 (0.20) |
| Day 28 | 0.0 (0.0) | 0.64 (0.09) | 0.71 (0.16) | 0.80 (0.21) |
| Change | 0.0 (0.0) | −0.14 (0.19) | −0.19 (0.25) | −0.05 (0.18) |
| p-value* | — | 0.15 | 0.05 | 0.42 |
| | Group 2 | | | |
| Day 1 | 0.0 (0.0) | 0.76 (0.20) | 0.86 (0.21) | 0.91 (0.13) |
| Day 28 | 0.0 (0.0) | 0.65 (0.15) | 0.69 (0.20) | 0.76 (0.01) |
| Change | 0.0 (0.0) | −0.17 (0.13) | −0.10 (0.29) | −0.12 (0.15) |
| p-value* | — | 0.15 | 0.62 | 0.30 |
| | Group 3 | | | |
| Day 1&28* | 0.0 (0.0) | 1.15 (0.49) | 0.95 (0.42) | 0.80 (0.26) |
| Day 28&1 | 0.0 (0.0) | 0.83 (0.07) | 0.91 (0.16) | 1.00 (0.10) |
| Change | 0.0 (0.0) | −0.33 (0.40) | −0.04 (0.56) | 0.20 (0.36) |
| p-value* | — | 0.45 | 0.92 | 0.44 |

Numbers in parentheses are standard deviations

For Group 1 (12 subjects), after supplementing with NoGlo, individuals saw an average reduction in blood acetaldehyde of 18.4%, 10 minutes after consuming alcohol. After 20 minutes the average reduction was 20.9%. After 40 minutes the average reduction was 5.4%.

For Group 2 (4 subjects, supplementing with NoGlo-2 supplement), after supplementing, individuals saw an average reduction in blood acetaldehyde of 22.4%, 10 minutes after consuming alcohol. After 20 minutes the average reduction was 11.6%. After 40 minutes the average reduction was 13.2%.

For Group 3 (4 subjects, supplementing with placebo followed by a single dose of the NoGlo supplement), after supplementing, individuals saw an average reduction in blood acetaldehyde of 28.7%, 10 minutes after consuming alcohol. After 20 minutes the average reduction was 4.2%. After 40 minutes there was an average increase in blood acetaldehyde concentration by 25%.

Daily supplementation with NoGlo and NoGlo-2 represented consistent and significant reductions in blood acetaldehyde following ethanol consumption, the cause of the damage and symptoms associated with the alcohol reaction (Alcohol Flush Reaction).

### Principal Investigator and Subject Reported Levels of Facial Flushing

Red flushing of the face and skin is caused by inflammation, which is caused by increased acetaldehyde in the blood. This is the most overtly obvious symptom of the alcohol reaction. The study Investigator and the subjects were asked to rate the intensity of redness the subject was experiencing on a scale of 1-10 at each time points baseline (time 0 minutes) and then at 10, 20 and 40 minutes following the consumption of 0.25 g of ethanol per Kg body weight. Table 3 represents the reported data by the Principle Investigator for facial flushing in the subject.

TABLE 3
Facial flushing reported by the study PI

| Flushing Scale | | | | |
|---|---|---|---|---|
| by PI | 0 min. | 10 min. | 20 min. | 40 min. |
| | Group 1 | | | |
| Day 1 | 1.3 (0.5) | 6.4 (2.0) | 9.0 (1.1) | 7.8 (1.9) |
| Day 28 | 1.3 (0.5) | 5.3 (1.6) | 7.7 (1.8) | 8.3 (2.3) |
| Change | 0.0 (0.7) | −1.1 (2.3) | −1.3 (1.5) | 0.5 (3.1) |
| p-value* | 1.00 | 0.13 | 0.01 | 0.58 |
| | Group 2 | | | |
| Day 1 | 1.3 (0.5) | 7.8 (1.7) | 9.5 (1.0) | 8.0 (0.8) |
| Day 28 | 1.3 (0.5) | 5.5 (0.6) | 8.5 (0.6) | 8.8 (1.0) |
| Change | 0.0 (0.8) | −2.3 (1.7) | −1.0 (1.4) | 0.8 (1.7) |
| p-value* | 1.00 | 0.08 | 0.25 | 0.44 |
| | Group 3 | | | |
| Day 1&28* | 1.0 (0.0) | 5.8 (3.2) | 8.0 (1.4) | 7.8 (1.5) |
| Day 28&1 | 1.3 (0.5) | 5.0 (0.0) | 8.5 (1.0) | 8.3 (2.1) |
| Change | 0.3 (0.5) | −0.8 (3.2) | 0.5 (1.7) | 0.5 (2.5) |
| p-value* | 0.39 | 0.67 | 0.60 | 0.72 |

Numbers in parentheses are standard deviations

For Group 1 (12 subjects), after supplementing with NoGlo, individuals saw an average reduction in reported facial flushing of 17.2%, 10 minutes after consuming alcohol. After 20 minutes the average reduction was 14.4%. After 40 minutes an average increase was reported of 6.4%.

For Group 2 (4 subjects, supplementing with NoGlo-2 supplement), after supplementing, individuals saw an average reduction in reported facial flushing of 29.5%, 10 minutes after consuming alcohol. After 20 minutes the average reduction was 10.5%. After 40 minutes an average increase was reported of 10%.

For Group 3 (4 subjects, supplementing with placebo followed by a single dose of the NoGlo supplement), after supplementing, individuals saw an average reduction in reported facial flushing of 13.8%, 10 minutes after consuming alcohol. After 20 minutes an average increase was reported of 6.3%. After 40 minutes an average increase was reported of 6.4%.

Daily supplementation with NoGlo and NoGlo-2 represented consistent reductions in facial flushing at 10 and 20 minutes following ethanol consumption.

Table 4 represents the self-reported data by the subject for the intensity of facial flushing.

TABLE 4
Facial flushing self-reported by the subject

| Flushing Scale | | | | |
|---|---|---|---|---|
| by Subject | 0 min. | 10 min. | 20 min. | 40 min. |
| | Group 1 | | | |
| Day 1 | 1.1 (0.3) | 5.1 (2.2) | 7.3 (2.2) | 6.6 (2.4) |
| Day 28 | 1.2 (0.6) | 4.5 (1.2) | 7.0 (1.6) | 7.6 (2.3) |
| Change | 0.1 (0.7) | −0.6 (1.7) | −0.3 (2.4) | 1.0 (3.6) |
| p-value* | 0.67 | 0.25 | 0.72 | 0.35 |
| | Group 2 | | | |
| Day 1 | 1.0 (0.0) | 6.8 (1.0) | 9.0 (0.5) | 7.8 (1.5) |
| Day 28 | 1.0 (0.0) | 4.5 (1.3) | 7.5 (1.7) | 8.5 (1.3) |
| Change | 0.0 (0.0) | −2.3 (0.5) | −1.5 (1.9) | 0.8 (1.7) |
| p-value* | — | 0.003 | 0.22 | 0.44 |
| | Group 3 | | | |
| Day 1&28* | 1.3 (0.5) | 5.0 (3.2) | 5.5 (3.7) | 5.5 (3.3) |
| Day 28&1 | 1.3 (0.5) | 5.5 (3.0) | 6.8 (2.5) | 7.3 (2.9) |
| Change | 0.0 (0.8) | 0.5 (4.8) | 1.3 (5.0) | 1.8 (5.6) |
| p-value* | 1.00 | 0.85 | 0.65 | 0.57 |

Numbers in parentheses are standard deviations

For Group 1 (12 subjects), after supplementing with NoGlo, individuals saw an average reduction in reported facial flushing of 11.8%, 10 minutes after consuming alcohol. After 20 minutes the average reduction was 4.1%. After 40 minutes an average increase was reported of 15.2%.

For Group 2 (4 subjects, supplementing with NoGlo-2 supplement), after supplementing, individuals saw an average reduction in reported facial flushing of 33.8%, 10 minutes after consuming alcohol. After 20 minutes the average reduction was 16.7%. After 40 minutes an average increase was reported of 10.3%.

For Group 3 (4 subjects, supplementing with placebo followed by a single dose of the NoGlo supplement), after supplementing, individuals saw an average increase in reported facial flushing of 10%, 10 minutes after consuming alcohol. After 20 minutes an average increase was reported of 23.6%. After 40 minutes an average increase was reported of 32.7%.

Daily supplementation with NoGlo and NoGlo-2 resulted in consistent reductions in facial flushing at 10 and 20 minutes following ethanol consumption.

### Self-Reported Physical Experience

The alcohol reaction is a combination of negative physical symptoms. Dizziness, nausea, headache, lightheadedness, feeling of overheating, and general physical discomfort, are all recognized as symptoms of the alcohol reaction. The subjects were asked to rate their physical experience for each of these symptoms on a scale of 1-10 after 40 minutes following the consumption of 0.25 g of ethanol per Kg body weight (Table 5).

TABLE 5
Self-reported physical experience ratings

| | Group 1 | Group 2 | Group 3 |
|---|---|---|---|
| Dizziness | | | |
| Day 1 | 3.6 (2.5) | 4.8 (2.6) | 5.3 (4.4) |
| Day 28 | 3.0 (2.4) | 4.0 (2.6) | 4.3 (2.6) |
| Change | −0.6 (1.1) | −0.8 (2.2) | −1.0 (3.8) |
| p-value* | 0.16 | 0.75 | 0.64 |
| Nausea | | | |
| Day 1 | 3.2 (2.2) | 3.0 (2.4) | 3.5 (3.3) |
| Day 28 | 2.8 (2.5) | 2.8 (2.4) | 2.8 (2.9) |
| Change | −0.3 (1.1) | −0.3 (2.1) | −0.8 (1.0) |
| p-value* | 0.44 | 1.00 | 0.22 |
| Headache | | | |
| Day 1 | 2.6 (1.8) | 3.8 (2.1) | 3.5 (2.4) |
| Day 28 | 2.7 (2.1) | 1.8 (1.0) | 3.3 (2.1) |
| Change | 0.1 (2.1) | −2.0 (1.4) | −0.3 (2.2) |
| p-value* | 0.96 | 0.25 | 0.84 |
| Lightheaded | | | |
| Day 1 | 4.3 (2.6) | 6.8 (2.2) | 4.8 (2.1) |
| Day 28 | 3.5 (2.6) | 4.0 (2.6) | 4.5 (2.5) |
| Change | −0.8 (2.0) | −2.8 (2.1) | −0.3 (1.7) |
| p-value* | 0.32 | 0.13 | 0.79 |
| Overheating | | | |
| Day 1 | 6.4 (2.7) | 7.5 (1.3) | 4.8 (3.3) |
| Day 28 | 4.9 (2.6) | 7.0 (0.8) | 5.0 (2.7) |
| Change | −1.5 (2.4) | −0.5 (0.6) | 0.3 (1.9) |
| p-value* | 0.06 | 0.50 | 0.81 |
| Uncomfortable/Unpleasant | | | |
| Day 1 | 4.8 (1.8) | 7.3 (1.7) | 4.8 (2.6) |
| Day 28 | 4.2 (2.4) | 7.3 (1.7) | 4.8 (3.3) |
| Change | −0.6 (2.2) | 0.0 (3.3) | 0.0 (2.8) |
| p-value* | 0.39 | 1.00 | 1.00 |

Numbers in parentheses are standard deviations

### Group 1 Change in Pre and Post Supplementation Experience:

Dizziness: Average reduction of 16.7%.

Nausea: Average reduction of 9.4%

Headache: Average increase of 3.4%

Lightheadedness: Average reduction of 18.6%

Overheating: Average reduction of 23.4%

Overall Discomfort: Average reduction of 12.5%

### Group 2 Change in Pre and Post Supplementation Experience:

Dizziness: Average reduction of 16.7%.

Nausea: Average reduction of 10%

Headache: Average increase of 52.6%

Lightheadedness: Average reduction of 41.2%

Overheating: Average reduction of 6.7%

Overall Discomfort: No average change was recorded

### Various Embodiments of the Invention

Additional embodiments of the invention are described as follows:

### Embodiment 1

A composition for use by a subject to prevent or reduce the alcohol reaction, said composition comprising:

a) an agent capable of increasing intracellular or intramitochondrial concentration of NAD in the subject;

b) an agent capable of increasing glutathione concentration in the subject;

c) an agent which is an anti-oxidant;

d) an agent which is alpha-lipoic acid or a precursor to alpha-lipoic acid; and

e) an agent capable of increasing the level of Coenzyme A in the subject;

wherein said agents are present in the composition in an amount effective to prevent or reduce the alcohol reaction in the subject.

### Embodiment 2

The composition of embodiment 1, wherein the agent capable of increasing intracellular or intramitochondrial concentration of NAD in the subject is nicotinamide.

### Embodiment 3

The composition of embodiment 2, wherein the amount of nicotinamide in the dosage is between about 500 mg to about 1,500 mg.

### Embodiment 4

The composition of any of embodiments 1-3, wherein the agent capable of increasing glutathione concentration in the subject is N-acetyl cysteine.

### Embodiment 5

The composition of embodiment 4, wherein the amount of N-acetyl cysteine in the dosage is between about 200 mg to about 600 mg.

### Embodiment 6

The composition of any of embodiments 1-5, wherein the anti-oxidant is Vitamin C.

### Embodiment 7

The composition of embodiment 6, wherein the amount of Vitamin C in the dosage is between about 100 mg to about 500 mg.

### Embodiment 8

The composition of any of embodiments 1-7, wherein the amount of alpha lipoic acid in the dosage is between about 50 mg to about 250 mg.

### Embodiment 9

The composition of any of embodiments 1-8, wherein the agent capable of increasing the level of Coenzyme A in the subject is pantothenic acid.

### Embodiment 10

The composition of embodiment 9, wherein the amount of pantothenic acid in the dosage is about 50 mg to about 250 mg.

### Embodiment 11

The composition of any of embodiments 1-9, further comprising an agent capable of raising the blood concentration of all-trans retinoic acid in a subject.

### Embodiment 12

A method of preventing or reducing at least one symptom of the alcohol reaction in a subject, comprising the step of administering to the subject a dosage of a composition according to any one of embodiments 1-11, or use of any composition according to embodiments 1-11 for preventing or reducing at least one symptom of the alcohol reaction in a subject.

### Embodiment 13

The method or use of embodiment 12, wherein the dosage is administered to the subject daily, either in a single dose per day, or in multiple divided doses per day.

### Embodiment 14

The method or use of embodiment 12 or embodiment 13, wherein the composition is administered to the subject for at least seven days prior to consumption of alcohol by the subject.

### Embodiment 15

The method or use of any of embodiments 12-14, wherein the at least one symptom is selected from the group consisting of facial flushing, hangover, dizziness, nausea, increased blood acetaldehyde concentration, and increased heart rate.

### Embodiment 16

The method or use of any of embodiments 12-15, wherein the at least one symptom is facial flushing.

### Embodiment 17

The method or use of any of embodiments 12-15, wherein the at least one symptom is hangover.

### Embodiment 18

The method or use of any of embodiments 12-15, wherein the at least one symptom is increased blood acetaldehyde concentration.

### Embodiment 19

The method or use of any of embodiments 12-15, wherein the at least one symptom is increased heart rate.

### Embodiment 20

The method or use of any of embodiments 12-15, wherein the at least one symptom is nausea.

### Embodiment 21

The method or use of any of embodiments 12-15, wherein the at least one symptom is dizziness.

### Embodiment 22

The composition of any of embodiments 1-11 for use in preventing or reducing at least one symptom of the alcohol reaction in a subject.

### Embodiment 23

Use of a composition of any of embodiments 1-11 for the manufacture of a supplement or medicament for use in preventing or reducing at least one symptom of the alcohol reaction in a subject.

### Embodiment 24

A composition comprising:

a) an agent capable of increasing intracellular or intramitochondrial concentration of NAD in a subject; and

b) an agent capable of increasing glutathione concentration in the subject.

### Embodiment 25

The composition of embodiment 24, wherein said agents are present in the composition in an amount effective to prevent or reduce the alcohol reaction in the subject.

### Embodiment 26

The composition of embodiment 24 or embodiment 25, further comprising at least one additional agent selected from the group comprising: an agent which is an anti-oxidant; an agent which is alpha-lipoic acid or a precursor to alpha-lipoic acid; and an agent capable of increasing the level of Coenzyme A in the subject.

### Embodiment 27

A composition comprising:

a) an agent capable of increasing intracellular or intramitochondrial concentration of NAD in a subject;

b) an agent capable of increasing glutathione concentration in the subject; and

c) an anti-oxidant.

### Embodiment 28

A composition comprising:

a) an agent capable of increasing intracellular or intramitochondrial concentration of NAD in a subject;

b) an agent capable of increasing glutathione concentration in the subject; and

c) alpha-lipoic acid or a precursor to alpha-lipoic acid.

### Embodiment 29

A composition comprising:

a) an agent capable of increasing intracellular or intramitochondrial concentration of NAD in a subject;

b) an agent capable of increasing glutathione concentration in the subject; and

c) an agent capable of increasing the level of Coenzyme A in the subject.

### Embodiment 30

A composition comprising:

a) an agent capable of increasing intracellular or intramitochondrial concentration of NAD in a subject;

b) an agent capable of increasing glutathione concentration in the subject;

c) an anti-oxidant; and

d) alpha-lipoic acid or a precursor to alpha-lipoic acid.

### Embodiment 31

A composition comprising:

a) an agent capable of increasing intracellular or intramitochondrial concentration of NAD in a subject;

b) an agent capable of increasing glutathione concentration in the subject;

c) an anti-oxidant; and

d) an agent capable of increasing the level of Coenzyme A in the subject.

### Embodiment 32

A composition comprising:

a) an agent capable of increasing intracellular or intramitochondrial concentration of NAD in a subject;

b) an agent capable of increasing glutathione concentration in the subject;

c) alpha-lipoic acid or a precursor to alpha-lipoic acid; and

d) an agent capable of increasing the level of Coenzyme A in the subject.

### Embodiment 33

The composition of any of embodiments 24-32, wherein said agents are present in the composition in an amount effective to prevent or reduce the alcohol reaction in the subject.

### Embodiment 34

A composition for use by a subject to prevent or reduce the alcohol reaction, said composition comprising:

a) an agent capable of increasing intracellular or intramitochondrial concentration of NAD in the subject;

b) an agent capable of increasing glutathione concentration in the subject;

c) an agent which is an anti-oxidant; and

d) an agent which is alpha-lipoic acid or a precursor to alpha-lipoic acid; and

e) an agent capable of increasing the level of Coenzyme A in the subject.

### Embodiment 35

The composition of any of embodiments 24-33 or the composition for use of embodiment 34, wherein said agents are present in the composition in an amount effective to prevent or reduce the alcohol reaction in a subject.

### Embodiment 36

The composition for use of any of embodiments 1-11 or 34, further comprising an agent capable of raising the blood level of all-trans retinoic acid.

### Embodiment 37

The composition of any of embodiments 24-33, further comprising an agent capable of raising the blood level of all-trans retinoic acid.

### Embodiment 38

The method or use of any of embodiments 12-23, wherein the composition, supplement, or medicament further comprises an agent capable of raising the blood level of all-trans retinoic acid.

### Embodiment 39

A composition comprising: a) an agent selected from the group consisting of nicotinamide, nicotinamide riboside, niacin, and oxaloacetate; and b) an agent selected from the group consisting of N-acetyl-cysteine, Vitamin C, alpha lipoic acid, L-cysteine, glutathione, glycine, L-glutamic acid, adenosylmethionine, and conjugated linoleic acid; wherein said agents are present in the composition in an amount sufficient to prevent or reduce the alcohol reaction in the subject.

### Embodiment 40

The composition of embodiment 39, further comprising an agent selected from the group consisting of pantothenic acid, pantethine, pantetheine, 4′-phosphopantothenate, and 4′-phosphopantetheine.

### Embodiment 38

The composition of embodiment 39 or embodiment 40, further comprising an agent which is selected from the group consisting of Vitamin C, Vitamin E, and alpha-lipoic acid.

### Embodiment 39

The composition of embodiment 36 or embodiment 37, further comprising alpha-lipoic acid or a precursor to alpha-lipoic acid.

### Embodiment 40

The composition of any one of embodiments 36-39, further comprising an agent selected from the group consisting of beta-carotene and a retinol ester.

### Embodiment 41

The composition of embodiment 40, wherein the agent is a retinol ester selected from the group consisting of retinyl arachidonate, retinyl stearate, retinyl oleate, retinyl linoleate, retinyl heptadecanoate, retinyl palmitate, retinyl myristate, retinyl laurate, retinyl caprylate, and retinyl acetate.

### Embodiment 42

A composition comprising an agent capable of increasing intracellular or intramitochondrial concentration of NAD in a subject; and Hovenia dulcis fruit extract.

### Embodiment 43

A composition comprising an agent capable of increasing intracellular or intramitochondrial concentration of NAD in a subject; and Silybum marianum extract.

### Embodiment 44

A composition comprising an agent capable of increasing intracellular or intramitochondrial concentration of NAD in a subject; Hovenia dulcis fruit extract; and Silybum marianum extract.

### Embodiment 45

A composition comprising: a) an agent selected from the group consisting of nicotinamide, nicotinamide riboside, niacin, and oxaloacetate; and b) one or more, and preferably, 3, 4, 5 or more, agents selected from the group consisting of Hovenia dulcis fruit extract, Silybum marianum extract, N-acetyl-cysteine, Vitamin C, alpha lipoic acid, L-cysteine, glutathione, glycine, L-glutamic acid, adenosylmethionine, and conjugated linoleic acid; wherein said agents are present in the composition in an amount sufficient to prevent or reduce the alcohol reaction in the subject.

### Embodiment 46

A composition comprising: a) an agent selected from the group consisting of nicotinamide, nicotinamide riboside, niacin, and oxaloacetate; b) Hovenia dulcis fruit extract; c) Silybum marianum extract; and d) α-lipoic acid; wherein said agents are present in the composition in an amount sufficient to prevent or reduce the alcohol reaction in the subject.

### Embodiment 47

A composition comprising: a) an agent selected from the group consisting of nicotinamide, nicotinamide riboside, niacin, and oxaloacetate; b) Hovenia dulcis fruit extract; c) Silybum marianum extract; d) vitamin A; e) vitamin B5; f) vitamin c; g) N-acetyl cysteine; h) thiamine; and i) α-lipoic acid; wherein said agents are present in the composition in an amount sufficient to prevent or reduce the alcohol reaction in the subject.

### Embodiment 48

A composition as described herein in Embodiments 42-47, comprising at least 500 mg niacinamide.

### Embodiment 49

Any of the compositions, compositions for use, or methods of Embodiments 1-47, wherein the composition, supplement, dosage, or medicament is in tablet or capsule form.

### Embodiment 50

Any of the compositions, compositions for use, or methods of Embodiments 1-47, wherein the composition, supplement, dosage, or medicament is in liquid or beverage form.

### Embodiment 51

Any of the compositions, compositions for use, or methods of Embodiments 1-47, wherein the composition, supplement, dosage, or medicament is provided in a food.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A composition comprising (i) between 300 mg and 1.5 g nicotinamide, (ii) between 10 mg and 500 mg pantothenic acid or a salt thereof, and (iii) between 100 mg and 600 mg N-acetylcysteine, and an excipient acceptable for daily oral administration.

2. The composition of claim 1, comprising (i) between 500 mg and 900 mg nicotinamide, (ii) between 25 mg and 75 mg pantothenic acid or a salt thereof, and (iii) between 200 mg and 400 mg N-acetylcysteine, and an excipient acceptable for daily oral administration.

3. A composition comprising (i) about 700 mg nicotinamide, (ii) about 50 mg pantothenic acid or a salt thereof, and (iii) about 300 mg N-acetylcysteine, and an excipient acceptable for daily oral administration, wherein the term “about” refers to +/−20% of each quantity.

4. A composition comprising (i) 700 mg nicotinamide, (ii) 50 mg pantothenic acid or a salt thereof, and (iii) 300 mg N-acetylcysteine, and an excipient acceptable for daily oral administration.

5. A composition according to claim 1, further comprising one or more of vitamin A, vitamin C, thiamine, and α-lipoic acid.

6. A composition according to claim 1, further comprising one or more of 1250 IU to 3750 IU vitamin A, 75 mg to 250 mg vitamin C, 2.5 to 100 mg thiamine, and 50 to 125 mg α-lipoic acid.

7. The composition of claim 5, comprising each of vitamin A, vitamin C, thiamine, and α-lipoic acid.

8. The composition of claim 7, comprising about 700 mg nicotinamide, about 50 mg pantothenic acid, about 300 mg N-acetylcysteine, about 2500 IU vitamin A, about 150 mg vitamin C, about 50 mg thiamine, and about 80 mg α-lipoic acid, wherein the term “about” refers to +/−20% of each quantity.

9. The composition of claim 8, comprising 700 mg nicotinamide, 50 mg pantothenic acid, 300 mg N-acetylcysteine, 2500 IU vitamin A, 150 mg vitamin C, 50 mg thiamine, and 80 mg α-lipoic acid.

10. The composition of claim 1, comprising (i) about 700 mg nicotinamide, (ii) about 50 mg pantothenic acid or a salt thereof, and (iii) between 300 mg and 600 mg N-acetylcysteine, and an excipient acceptable for daily oral administration, wherein the term “about” refers to +/−20% of each quantity.

11. The composition of claim 1, further comprising about 2500 IU vitamin A, 150 mg to 1,000 mg vitamin C, about 50 mg thiamine, and about 80 mg α-lipoic acid, wherein the term “about” refers to +/−20% of each quantity.

12. The composition of claim 1, wherein the pantothenic acid is provided in the form of calcium pantothenate.

13. A composition for use by a subject to prevent or reduce the alcohol reaction, said composition comprising:
a) an agent selected from the group consisting of nicotinamide, nicotinamide riboside, niacin, and oxaloacetate; and
b) an agent selected from the group consisting of N-acetyl-cysteine, Vitamin C, alpha lipoic acid, L-cysteine, glutathione, glycine, L-glutamic acid, adenosylmethionine, and conjugated linoleic acid;
wherein said agents are present in the composition in an amount sufficient to prevent or reduce the alcohol reaction in the subject.

14. The composition of claim 13, further comprising an agent selected from the group consisting of pantothenic acid, pantethine, pantetheine, 4′-phosphopantothenate, and 4′-phosphopantetheine.

15. The composition of claim 13, further comprising an agent which is selected from the group consisting of Vitamin C, Vitamin E, and alpha-lipoic acid.

16. The composition of claim 13, further comprising alpha-lipoic acid or a precursor to alpha-lipoic acid.

17. The composition of claim 13, further comprising an agent selected from the group consisting of beta-carotene and a retinol ester.

18. The composition of claim 17, wherein the agent is a retinol ester selected from the group consisting of retinyl arachidonate, retinyl stearate, retinyl oleate, retinyl linoleate, retinyl heptadecanoate, retinyl palmitate, retinyl myristate, retinyl laurate, retinyl caprylate, and retinyl acetate.

19. A method of reducing the alcohol reaction in a subject, comprising administering a composition of claim 1 to the subject daily for a period of at least two weeks prior to consumption of alcohol by the subject.

20. The method of claim 19, wherein the composition is administered orally.

21. The method of claim 19, wherein the composition is administered to the subject daily for a period of at least four weeks.

22-24. (canceled)

25. A method of preventing or reducing at least one symptom of the alcohol reaction in a subject, comprising administering to the subject at least one agent selected from the group consisting of:
an agent capable of increasing intracellular or intramitochondrial concentration of NAD in the subject, an agent capable of increasing glutathione concentration in the subject, and an agent capable of increasing cellular Coenzyme A concentrations in the subject;
wherein the at least one agent is administered in an amount sufficient to prevent or reduce the alcohol reaction in the subject.

26. The method of claim 25, wherein the dosage is administered to the subject daily, either in a single dose per day, or in multiple divided doses per day.

27. The method of claim 25, wherein the composition is administered to the subject daily for at least seven days prior to consumption of alcohol by the subject.

28. The method of claim 25, wherein the at least one symptom is selected from the group consisting of facial flushing, hangover, dizziness, nausea, increased blood acetaldehyde concentration, and increased heart rate.

29. A method of reducing blood levels of acetaldehyde resulting from dietary ethanol consumption by a human adult, comprising orally administering a daily dosage of
(i) between 300 mg and 1.5 g nicotinamide,
(ii) between 10 mg and 500 mg pantothenic acid or a salt thereof, and
(iii) between 100 mg and 600 mg N-acetylcysteine for a period of time selected from the group consisting of at least 3 days, 5 days, 10 days, 14 days, 21 days, and 28 days.

30. The method of claim 29, wherein the daily dosage comprises about 700 mg nicotinamide, about 50 mg pantothenic acid, and about 300 mg N-acetylcysteine, wherein the term “about” refers to +/−20% of each quantity.

31. The method of claim 29, wherein the daily dosage comprises 700 mg nicotinamide, 50 mg pantothenic acid, and 300 mg N-acetylcysteine.

32. The method of claim 29, wherein the nicotinamide, pantothenic acid, and N-acetylcysteine are provided as a tablet or capsule.

33. The method of claim 29, wherein the nicotinamide, pantothenic acid, and N-acetylcysteine are provided as a beverage.

34. The method of claim 29, wherein the nicotinamide, pantothenic acid, and N-acetylcysteine are provided as a food.

35. The method of claim 29, wherein the pantothenic acid is provided in the form of calcium pantothenate.

36. The method of claim 29, further comprising administering a dosage of (i) between 400 mg and 1.5 g nicotinamide, (ii) between 10 mg and 500 mg pantothenic acid, and between 100 mg and 600 mg N-acetylcysteine to the human adult between 10 minutes and 1 hour prior to consumption of ethanol by the human adult.

37. The method of claim 29, wherein the human adult has an E487K mutation in mitochondrial aldehyde dehydrogenase and/or a R47H mutation in alcohol dehydrogenase 1B.

38. The method of claim 29, wherein the levels of nicotinamide, pantothenic acid, and N-acetylcysteine obtained after 30 days are sufficient to reduce average peak blood levels of acetaldehyde in a population of human adults having an E487K mutation in mitochondrial aldehyde dehydrogenase and/or a R47H mutation in alcohol dehydrogenase 1B following ingestion of at least 0.3 g ethanol/kg body weight by at least 5% relative to average peak blood levels in a corresponding untreated population.

39. The method of claim 29, wherein the daily dosage further comprises one or more of vitamin A, vitamin C, thiamine, and α-lipoic acid.

40. The method of claim 39, wherein the daily dosage further comprises one or more of 1250 IU to 3750 IU vitamin A, 75 mg to 250 mg vitamin C, 2.5 to 100 mg thiamine, and 50 to 125 mg α-lipoic acid.

41. The method of claim 39, wherein the daily dosage further comprises each of vitamin A, vitamin C, thiamine, and α-lipoic acid.

42. A composition for use by a subject to prevent or reduce the alcohol reaction, said composition comprising:
a) an agent capable of increasing intracellular or intramitochondrial concentration of NAD in the subject; and
b) an agent capable of increasing glutathione concentration in the subject;
wherein said agents are present in the composition in an amount sufficient to prevent or reduce the alcohol reaction in the subject.

43. The composition of claim 42, further comprising an agent capable of increasing the level of Coenzyme A in the subject.

44. The composition of claim 43, further comprising an agent which is an anti-oxidant.

45. The composition of claim 44, further comprising alpha-lipoic acid or a precursor to alpha-lipoic acid.

46. The composition of claim 45, further comprising an agent capable of raising the blood level of all-trans retinoic acid.

47. The composition of claim 42, wherein the agent capable of increasing intracellular or intramitochondrial concentration of NAD in the subject is selected from the group consisting of nicotinamide, nicotinamide riboside, niacin, and oxaloacetate.

48. The composition of claim 42, wherein the agent capable of increasing glutathione concentration in the subject is selected from the group consisting of N-acetyl-cysteine, Vitamin C, alpha lipoic acid, L-cysteine, glutathione, glycine, L-glutamic acid, adenosylmethionine, and conjugated linoleic acid.

49. The composition of claim 43, wherein the agent capable of increasing the level of Coenzyme A in the subject is selected from the group consisting of pantothenic acid, pantethine, pantetheine, 4′-phosphopantothenate, and 4′-phosphopantetheine.

50. The composition of claim 44, wherein the anti-oxidant is Vitamin C.

51. A method of preventing or reducing at least one symptom of the alcohol reaction in a subject, comprising administering to the subject a dosage of a composition according to claim 42.

52. The method of claim 51, wherein the dosage is administered to the subject daily, either in a single dose per day, or in multiple divided doses per day.

53. The method of claim 51, wherein the composition is administered to the subject daily for at least seven days prior to consumption of alcohol by the subject.

54. The method of claim 51, wherein the at least one symptom is selected from the group consisting of facial flushing, hangover, dizziness, nausea, increased blood acetaldehyde concentration, and increased heart rate.
